(19) United States
(12) Patent Application Publication
(10) Pub. No.: US 2023/0404968 A1
(43) Pub. Date: Dec. 21, 2023

(54) Gamma Butyrolactone Derivatives for Inducing Differentiation in Neoplastic Cells

(71) Applicant: Amir Salehi-Najafabadi, Tehran (IR)

(72) Inventors: Sepand Tehrani Fateh, Tehran (IR); Sahand Tehrani Fateh, Tehran (IR); Amir Salehi-Najafabadi, Tehran (IR)

(21) Appl. No.: 18/336,744

(22) Filed: Jun. 16, 2023

Related U.S. Application Data
(60) Provisional application No. 63/353,756, filed on Jun. 20, 2022.

(51) Int. Cl. A61K 31/365; A61P 35/02; G01N 33/50

(52) U.S. Cl. CPC A61K 31/365; A61P 35/02; G01N 33/5011

(57) ABSTRACT

Gamma butyrolactone derivatives for inducing differentiation in a neoplastic cell and methods for inducing differentiation in a neoplastic cell by contacting the neoplastic cell with a subapoptotic concentration of a gamma butyrolactone derivative are provided.

### CROSS-REFERENCE TO RELATED APPLICATIONS

The present application claims priority from U.S. Provisional Application No. 63/353,756 filed Jun. 20, 2022, the contents of which are hereby incorporated by reference in their entirety.

### TECHNICAL FIELD

The present invention relates in general to gamma butyrolactone derivatives for the induction of differentiation in neoplastic cells, and to methods of differentiation therapy using gamma butyrolactone derivatives at subapoptotic doses.

### BACKGROUND OF THE INVENTION

Cell differentiation is a complexly regulated process by which cells change their functional or phenotypical type, becoming more specialized. Cell differentiation is characterized by morphological, molecular, and functional features.

Tissues are composed of various cells, the cells being in different stages of cell differentiation. Terminally differentiated cells (fully differentiated cells) are cells that have transitioned from an immature and specialized state to genetically, functionally, and morphologically specialized state. Terminally differentiated cells perform specialized tissue functions. Stem cells, which are undifferentiated, possess an intrinsically low proliferation rate, but are capable of self-renewal and of giving rise to daughter cells committed into various tissue-lineages.

Pathological loss of cell differentiation can lead to neoplastic cells, e.g., malignant cells and cancer. Many cancers have demonstrated poor cellular differentiation, differentiation block, or differentiation arrest, which are correlated with the aggressiveness of malignancies. Skin cancers, hepatocellular carcinoma, childhood cancers (e.g., Wilms), and leukemia, for example, are known to result from poor cellular differentiation. Evidence of the involvement of poor cellular differentiation, or of cancer stem cells, in carcinogenesis has also been observed in other cancers. For instance, it has been shown that breast cancers can arise from undifferentiated suprabasal progenitor cells above the myoepithelial cells in the duct or the terminal ductular lobular unit. Prostate cancer can also arise from suprabasal stem cells. Moreover, mutations in the adenomatous polyposis coli gene (APC) allows expansion of crypt stem cells and further development of colon cancer.

A novel and potentially less toxic form of neoplasm therapy, e.g., cancer therapy, called differentiation therapy, involves the use of agents that modify the state of differentiation of cancer cells to induce malignant reversion (i.e., the malignant phenotype becomes benign) and eliminate tumor phenotype. This therapeutic approach is based on the observation that many cancer subtypes possess a failure in the normal processes of differentiation, causing poor cellular differentiation that can be corrected by appropriate treatment. Through this therapeutic approach, the malignant self-replication of cancer cells is terminated by re-engaging cell differentiation in dedifferentiated or differentiation-arrested cells through reactivation of endogenous differentiation pathways. For example, the acute myeloid leukemia (AML) cell line HL60 terminally differentiates when treated with retinoids, sodium butyrate, cAMP, interferons, or DNA-demethylating agents. The doses of these compounds that are required to induce cellular differentiation are low enough so as not to impose cytotoxic effects, such as apoptosis or necrosis, on the treated cells.

### SUMMARY OF THE EMBODIMENTS

In accordance with one embodiment of the invention, a method for treating a human having a neoplasm, the method comprising:
contacting a neoplastic cell of the human, the neoplastic cell being associated with the neoplasm, with a subapoptotic concentration of a gamma butyrolactone derivative, or salt thereof, that induces differentiation in the neoplastic cell, the gamma butyrolactone derivative having the formula:

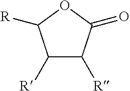

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

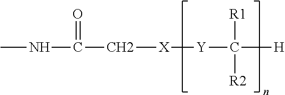

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
      (i) if Y is a single bond, R1 and R2 are each present,
      (ii) if Y is a double bond, R1 is present and R2 is absent, and
      (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

In accordance with another embodiment of the invention, a method for inducing differentiation in a neoplastic cell, the neoplastic cell being associated with a neoplasm, the method comprising:
contacting the neoplastic cell with a subapoptotic concentration of a gamma butyrolactone derivative, or salt thereof, that induces differentiation in the cancer cell, the gamma butyrolactone derivative having the formula:

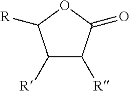

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

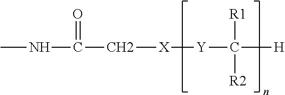

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
      (i) if Y is a single bond, R1 and R2 are each present,
      (ii) if Y is a double bond, R1 is present and R2 is absent, and
      (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

In accordance with one embodiment of the invention, a method for treating a human having cancer, the method comprising:
contacting a cancer cell of the human with a subapoptotic concentration of a gamma butyrolactone derivative, or salt thereof, that induces differentiation in the cancer cell, the gamma butyrolactone derivative having the formula:

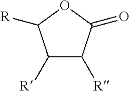

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

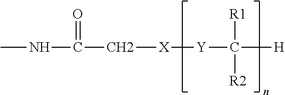

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
      (i) if Y is a single bond, R1 and R2 are each present,
      (ii) if Y is a double bond, R1 is present and R2 is absent, and
      (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

In accordance with another embodiment of the invention, a method for inducing differentiation in a cancer cell, the method comprising:
contacting the cancer cell with a subapoptotic concentration of a gamma butyrolactone derivative, or salt thereof, that induces differentiation in the cancer cell, the gamma butyrolactone derivative having the formula:

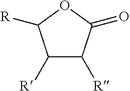

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

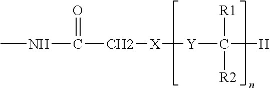

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
      (i) if Y is a single bond, R1 and R2 are each present,
      (ii) if Y is a double bond, R1 is present and R2 is absent, and
      (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

In some embodiments, a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

In other embodiments, R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and none of R, R′, and R″ are N-homoserine.

In some embodiments, the gamma butyrolactone derivative is HSL-C8, HSL-C12, A-factor, or GBL.

In some embodiments, the cancer is selected from the group consisting of sarcomas, carcinomas, and leukemias. In some embodiments, the cancer is selected from the group consisting of fibrosarcoma, myxosarcoma, liposarcoma, chondrosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystandenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bluffer carcinoma, epithelial carcinoma, glioma, astrocytomoma, pinealoma, hemangioblastoma, acoustic neuroma, oligodendroglioma, meningioma, melanoma, neuroblastoma, and retinoblastoma.

In some embodiments, the cancer is myeloid leukemia. The myeloid leukemia may be acute promyelocytic leukemia or acute myeloid leukemia.

In some embodiments, the induced differentiation is marked by an increase in the expression of a gene selected from the group consisting of FLT3, PU1, CEBPA, and combinations thereof.

In some embodiments, the induced differentiation is marked by an increase in the number of cancer cells expressing a marker selected from the group consisting of CD3, CD20, CD24, CD33, CD16, CD56, and combinations thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing features of embodiments will be more readily understood by reference to the following detailed description, taken with reference to the accompanying drawings, in which:

FIG. 1A shows a bar chart demonstrating the viability of peripheral blood mononuclear cells (PBMC) after treatment with 0, 25, 50, 100, 150, 200, 250, and 300 μM of HSL-C8 for 24, 48, and 72 hours as measured by MTS (3-(4,5-dimethylthiazol-2-yl)-5-(3-carboxymethoxyphenyl)-2-(4-sulfophenyl)-2H-tetrazolium), in accordance with embodiments of the invention. The relative toxicity of HSL-C8 was evaluated using an MTS assay after the treatment of cells with different concentrations of HSL-C8 for specific durations. FIG. 1B shows a bar chart demonstrating the viability of HL60 cells after treatment with 0, 25, 50, 100, 150, 200, 250, and 300 μM of HSL-C8 for 24, 48, and 72 hours as measured by MTS. FIG. 1C shows a bar chart demonstrating the viability of KG1a cells after treatment with 0, 25, 50, 100, 150, 200, 250, and 300 μM of HSL-C8 for 24, 48, and 72 hours as measured by MTS.

FIG. 2A shows a bar chart demonstrating the viability of PBMC cells after treatment with 0, 25, 50, 100, 150, 200, 250, and 300 μM of HSL-C12 for 24, 48, and 72 hours as measured by MTS, in accordance with embodiments of the invention. The relative toxicity of HSL-C12 was evaluated using an MTS assay after the treatment of cells with different concentrations of HSL-C12 for specific durations. FIG. 2B shows a bar chart demonstrating the viability of HL60 cells after treatment with 0, 25, 50, 100, 150, 200, 250, and 300 μM of HSL-C12 for 24, 48, and 72 hours as measured by MTS. FIG. 2C shows a bar chart demonstrating the viability of KG1a cells after treatment with 0, 25, 50, 100, 150, 200, 250, and 300 μM of HSL-C12 for 24, 48, and 72 hours as measured by MTS.

FIG. 3A shows scatter plots demonstrating minimal to no apoptosis (early and late) and necrosis for HL60 cells treated with 0, 25, 50, and 100 μM of HSL-C8 for 48 hours, in accordance with embodiments of the invention. The effect of HSL-C8 on apoptosis and necrosis of treated cells were evaluated by flow cytometry with PI/Anexxin-V-FITC staining.

FIG. 3B shows scatter plots demonstrating minimal to no apoptosis (early and late) and necrosis for HL60 cells treated with 0, 25, 50, and 100 μM of HSL-C8 for 72 hours. FIG. 3C shows scatter plots demonstrating minimal to no apoptosis (early and late) and necrosis for KG1a cells treated with 0, 25, 50, and 100 μM of HSL-C8 for 48 hours. FIG. 3D shows scatter plots that demonstrate minimal to no apoptosis (early and late) and necrosis for KG1a cells treated with 25, 50, and 100 μM of HSL-C8 for 72 hours.

FIG. 4A shows scatter plots demonstrating minimal to no apoptosis (early and late) and necrosis of HL60 cells treated with 0, 50, and 100 μM of HSL-C12 for 24 hours, in accordance with embodiments of the invention. The effect of HSL-C12 on apoptosis and necrosis of treated cells were evaluated by flow cytometry with PI/Anexxin-V-FITC staining.

FIG. 4B shows scatter plots demonstrating minimal to no apoptosis (early and late) and necrosis of HL60 cells treated with 0, 50, and 100 μM of HSL-C12 for 48 hours. FIG. 4C shows scatter plots demonstrating minimal to no apoptosis (early and late) and necrosis of KG1a cells treated with 100 μM of HSL-C12 for 24 hours and minimal apoptosis and necrosis of KG1a cells treated with 150 μM of HSL-C12 for 24 hours. FIG. 4D shows scatter plots demonstrating minimal to no apoptosis (early and late) and necrosis of KG1a cells treated with 100 μM of HSL-C12 for 48 hours and moderate apoptosis (early and late) and necrosis of KG1a cells treated with 150 μM of HSL-C12 for 48 hours. FIG. 4E shows scatter plots of positive control experiments demonstrating apoptosis of HL60 and KG1a cells treated with 200 μM of H₂O₂ for 24 hours and 72 hours.

FIG. 5A shows a bar chart illustrating FLT3, PU1, and CEBPA gene expression, as determined by RT-PCR, for HL60 cells after treatment with 0, 25, 50, and 100 μM of HSL-C8 for 48 and 72 hours, in accordance with embodiments of the invention. FIG. shows a bar chart illustrating CD3, CD20, CD24, CD33, CD16, and CD56 marker expression, as determined by flow cytometry, for HL60 cells after treatment with 0, 25, 50, and 100 μM of HSL-C8 for 48 and 72 hours. The expression of FLT3, PU1, and CEBPA genes, and CD3, CD20, CD24, CD33, CD16, and CD56 markers, show the differentiation of poorly differentiated cancer cells from myeloid origin to differentiated cells of myeloid lineage, such as monocytes and neutrophils. FIG. 5C shows a bar chart illustrating FLT3, PU1, and CEBPA gene expression, as determined by RT-PCR, for KG1a cells after treatment with 0, 100, and 200 μM of HSL-C8 for 48 and 72 hours. FIG. 5D shows a bar chart illustrating CD3, CD20, CD24, CD33, CD16, and CD56 marker expression, as determined by flow cytometry, for KG1a cells after treatment with 0, 100, and 200 μM of HSL-C8 for 48 and 72 hours.

FIG. 6A shows a bar chart illustrating FLT3, PU1, and CEBPA gene expression, as determined by RT-PCR, for HL60 cells after treatment with 0, 50, and 100 μM of HSL-C12 for 24 and 48 hours, in accordance with embodiments of the invention.

FIG. 6B shows a bar chart illustrating CD3, CD20, CD24, CD33, CD16, and CD56 marker expression, as determined by flow cytometry, for HL60 cells after treatment with 0, 50, and 100 μM of HSL-C12 for 24 and 48 hours. The expression of FLT3, PU1, and CEBPA genes, and CD3, CD20, CD24, CD33, CD16, and CD56 markers, show the differentiation of poorly differentiated cancer cells from myeloid origin to differentiated cells of myeloid lineage, such as monocytes and neutrophils. FIG. 6C shows a bar chart illustrating PU1 and CEBPA gene expression, as determined by RT-PCR, for KG1a cells after treatment with 0, 50, and 100 μM of HSL-C12 for 24 and 48 hours. FIG. 6D shows a bar chart illustrating CD3, CD20, CD24, CD33, CD16, and CD56 marker expression, as determined by flow cytometry, for KG1a cells after treatment with 0, 50, and 100 μM of HSL-C12 for 24 and 48 hours.

FIG. 7A shows images obtained using Wright-Giemsa staining followed by light microscopy, showing morphological changes to HL60 cells treated with 0, 50, and 100 μM of HSL-C8 for 48 and 72 hours, in accordance with embodiments of the invention. FIG. 7B shows images obtained using Wright-Giemsa staining followed by light microscopy, showing morphological changes to KG1a cells treated with 0, 100, and 200 μM of HSL-C8 for 48 and 72 hours.

FIG. 8A shows images obtained using Wright-Giemsa staining followed by light microscopy, showing morphological changes to HL60 cells treated with 0, 100, and 150 μM of HSL-C12 for 24 and 48 hours, in accordance with embodiments of the invention. FIG. 8B shows images obtained using Wright-Giemsa staining followed by light microscopy, showing morphological changes to KG1a cells treated with 0, 100, and 150 μM of HSL-C12 for 24 and 48 hours.

FIG. 9A shows a bar chart demonstrating the viability of peripheral blood mononuclear cells (PBMC) after treatment with 0, 40, 80, 120, 160, and 200 μM of A-factor for 24, 48, and 72 hours as measured by MTS, in accordance with embodiments of the invention. The relative toxicity of A-factor was evaluated using an MTS assay after the treatment of cells with different concentrations of A-factor for specific durations. FIG. 9B shows a bar chart demonstrating the viability of HL60 cells after treatment with 0, 40, 80, 120, 160, and 200 μM of A-factor for 24, 48, and 72 hours as measured by MTS. FIG. 9C shows a bar chart demonstrating the viability of KG1a cells after treatment with 0, 40, 80, 120, 160, and 200 μM of A-factor for 24, 48, and 72 hours as measured by MTS.

FIG. 10A shows a bar chart demonstrating the viability of peripheral blood mononuclear cells (PBMC) after treatment with 0, 100, 200, 300, 400, and 500 μM of GBL for 24, 48, and 72 hours as measured by MTS, in accordance with embodiments of the invention. The relative toxicity of GBL was evaluated using an MTS assay after the treatment of cells with different concentrations of GBL for specific durations. FIG. 10B shows a bar chart demonstrating the viability of HL60 cells after treatment with 0, 100, 200, 300, 400, and 500 μM of GBL for 24, 48, and 72 hours as measured by MTS. FIG. 10C shows a bar chart demonstrating the viability of KG1a cells after treatment with 0, 100, 200, 300, 400, and 500 μM of A-factor for 24, 48, and 72 hours as measured by MTS.

FIG. 11 shows scatter plots demonstrating minimal to no apoptosis (early and late) and necrosis for HL60 and KG1a cells treated with 120 μM of A-factor and 300 μM of GBL for 48 hours, in accordance with embodiments of the invention. The effect of A-factor and GBL on apoptosis and necrosis of treated cells were evaluated by flow cytometry with PI/Anexxin-V-FITC staining.

FIG. 12A shows a bar chart illustrating FLT3, PU1, and CEBPA gene expression, as determined by RT-PCR, for HL60 cells after treatment with 0, 40, and 120 μM of A-factor for 24 and 48 hours, in accordance with embodiments of the invention. FIG. 12B shows a bar chart illustrating CD3, CD20, CD24, CD33, CD16, and CD56 marker expression, as determined by flow cytometry, for HL60 cells after treatment with 0, 40, and 120 μM of A-factor for 24 and 48 hours. The expression of FLT3, PU1, and CEBPA genes, and CD3, CD20, CD24, CD33, CD16, and CD56 markers, show the differentiation of poorly differentiated cancer cells from myeloid origin to differentiated cells of myeloid lineage, such as monocytes and neutrophils. FIG. 12C shows a bar chart illustrating FLT3, PU1, and CEBPA gene expression, as determined by RT-PCR, for KG1a cells after treatment with 0, and 120 μM of A-factor for 24 and 48 hours. FIG. 12D shows a bar chart illustrating CD3, CD20, CD24, CD33, CD16, and CD56 marker expression, as determined by flow cytometry, for KG1a cells after treatment with 0, 40, and 120 μM of A-factor for 24 and 48 hours.

FIG. 13A shows a bar chart illustrating FLT3, PU1, and CEBPA gene expression, as determined by RT-PCR, for HL60 cells after treatment with 0, 100, and 300 μM of GBL for 24 and 48 hours, in accordance with embodiments of the invention. FIG. 13B shows a bar chart illustrating CD3, CD20, CD24, CD33, CD16, and CD56 marker expression, as determined by flow cytometry, for HL60 cells after treatment with 0, 100, and 300 μM of GBL for 24 and 48 hours. The expression of FLT3, PU1, and CEBPA genes, and CD3, CD20, CD24, CD33, CD16, and CD56 markers, show the differentiation of poorly differentiated cancer cells from myeloid origin to differentiated cells of myeloid lineage, such as monocytes and neutrophils. FIG. 13C shows a bar chart illustrating FLT3, PU1, and CEBPA gene expression, as determined by RT-PCR, for KG1a cells after treatment with 0, 100, and 300 μM of GBL for 24 and 48 hours. FIG. 13D shows a bar chart illustrating CD3, CD20, CD24, CD33, CD16, and CD56 marker expression, as determined by flow cytometry, for KG1a cells after treatment with 0, 100, and 300 μM of A-GBL for 24 and 48 hours.

### DETAILED DESCRIPTION OF SPECIFIC EMBODIMENTS

As used herein, “neoplasm” means a new and abnormal growth of tissue occurring in a part of a mammalian body, e.g., a human body, and includes both malignant neoplasms, i.e., cancer, and benign neoplasms.

A “neoplastic cell” is a cell associated with a particular neoplasm, e.g., a cancer cell associated with a particular cancer type, or a benign neoplastic cell associated with a particular benign neoplasm. A neoplastic cell may exist in vivo in the mammalian body, be cultured outside of the mammalian body in vitro, or treated outside of the mammalian body ex vivo.

Many distinct pathways are involved in the process of differentiation that can be induced specifically via targeted differentiation inducing agents. The mechanism of action, effective dose, and application of differentiation inducing agents is distinct from those of agents used in other cancer therapeutic strategies, i.e., not all cancer treatment agents induce cellular differentiation. Induction of differentiation in cancer cells is distinguished from induction of differentiation in normal (non-cancer) cells because each has different characteristics. Cancer cells have been pathologically reprogrammed to become undifferentiated, resist against physiological differentiation-inducing signals, and possess malignant features, while normal non-cancer cells possess an intrinsic tendency to differentiate under physiological conditions.

Differentiation therapy possesses certain advantages over conventional cancer therapeutic methods. Cytotoxic chemotherapy aims to kill cancer cells, imposing cytotoxic effects to normal cells as well as cancer cells. The therapeutic effectiveness of conventional chemotherapy can be compromised due to the development of drug resistance (both single and multi-drug) and systemic toxicity. Moreover, conventional approaches (e.g., conventional cytotoxic agents, targeted antibodies or small molecule inhibitors) are not sufficient in effecting cures for a significant proportion of cancer patients.

Differentiation therapy, on the other hand, aims to transition cancer cells into normal cells, while minimizing cytotoxic effects on cancer cells as well as normal cells. Differentiation therapy offers a unique approach to target and treat a sub-population of cancers, specifically those with a differentiation block. This approach as a targeted therapy leads to lower systemic toxicity and reduced adverse effects. The therapeutic doses of compounds used for differentiation therapy are high enough to induce cellular differentiation, while not high enough to impose significant cytotoxic effects leading to apoptosis or necrosis in either cancer cells or normal cells. Lower adverse effects lead to improved compliance of patients to such treatment. As a result of differentiation therapy, normal cells are preserved, maintaining their normal physiological role, while cancer cells are transformed into differentiated functional cells.

Because some cancers can develop resistance to current differentiation therapy agents, e.g., retinoids, there is a need for the discovery of additional agents for use in differentiation therapy. These additional agents may also be useful in multi-drug treatment regimens and for more personalized treatment options.

Gamma butyrolactone (GBL), a 4-carbon lactone, is a butan-4-olide that is tetrahydrofuran substituted by an oxo group at position 2:

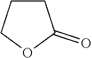

Homoserine lactones (HSLs) are one type of GBL derivative wherein GBL is bonded to an acyl chain with an amide bond (N-homoserine). Some HSLs originate from bacteria. In addition, some bacteria, specifically Streptomyces griseus, are capable of producing GBL derivatives such as A-factor (2-isocapryloyl-3R-hydroxymethyl-gamma-butyrolactone). HSLs have been shown to affect eukaryotic cells in various ways. For example, it has been shown that HSLs affect signaling pathways in human and animal cells. In addition, HSLs affect the energetics of human and animal cells and lead to an arrest in the cell cycle. At high enough concentrations, HSLs can cause apoptosis in human and animal cells. Moreover, HSLs have been shown to induce cell differentiation in precursor normal and healthy cells, e.g., osteoblasts and fibroblasts, without any defect in differentiation-related pathways.

The use of HSLs as cytotoxic apoptosis-inducing and cell growth-inhibiting agents (as opposed to use as a differentiation-inducing agent) in the elimination cancer cells has been demonstrated, and leads to the activation of cell death related pathways with cytotoxic effects on both cancerous and normal (non-cancer) cells, making such use of HSLs undesirable for treatment of cancer. HSLs have been shown to possess cancer cell growth inhibitory effects. Moreover, HSLs can modulate the activity of STAT for the treatment of a range of diseases, including cancer. Administration of a combination of HSLs and TRAIL (Tumor necrosis factor-related apoptosis-inducing ligand) to patients has been shown to inhibit cellular proliferation, arrest the cell cycle, and induce apoptosis in cancer cells. HSLs growth inhibiting effects routes in an arrest in the cell cycle and possible apoptosis, while for the induction of differentiation, more distinct pathways are specifically needed to be activated.

Embodiments of the invention described herein relate to the surprising finding that GBL derivatives can induce differentiation of cancer cells at concentrations that do not cause significant apoptosis (subapoptotic concentrations), offering a promising and less toxic treatment for cancers arising from poorly differentiated cells.

In some embodiments, the invention provides GBL derivatives for the induction of differentiation in neoplastic cells, and methods for inducing the differentiation of neoplastic cells in a patient (differentiation therapy). In some embodiments, these GBL derivatives can be used for differentiation therapy by contacting neoplastic cells with a subapoptotic concentration of at least one GBL derivative. For example, differentiation therapy using GBL derivatives may be used to treat poorly differentiated cancer cells such as cells associated with myeloid leukemia, including acute promyelocytic leukemia, acute myeloid leukemia, and chronic myelogenous leukemia.

In some embodiments, the invention provides GBL derivatives for eliminating or reducing a number of neoplastic cells, e.g., cancer cells, through malignant reversion (i.e., the reversion of a cancer cell from its relatively undifferentiated state to a differentiated state) while imposing minimal harmful effects to healthy and normal cells.

In some embodiments, GBL derivatives can be used for differentiation therapy of blood and lymphatic system cancers (e.g., acute lymphoblastic leukemia, chronic lymphoblastic leukemia, acute myeloid leukemia, acute promyelocytic leukemia, chronic myeloid leukemia, Hodgkin lymphoma, non-hodgkin lymphoma, diffuse large B-cell lymphoma, lymphangiosarcoma, lymphangioendotheliosarcoma, endotheliosarcoma), musculoskeletal cancers (e.g., synovial carcinoma, chondrosarcoma, Ewing's tumor, leiomyosarcoma, myosarcoma, rhabdomyosarcoma, liposarcoma, fibrosarcoma), gastrointestinal cancers (e.g., esophageal carcinoma, pancreatic adenocarcinoma, stomach adenocarcinoma, colon adenocarcinoma, bile duct carcinoma), breast cancers (e.g., ductal carcinoma, lobular carcinoma, tubular/cribriform carcinoma, mucinous carcinoma, medullary carcinoma, papillary carcinoma), skin cancers (e.g., squamous cell carcinoma, basal cell carcinoma, melanoma, sweat gland carcinoma, sebaceous gland carcinoma), thyroid cancers (papillary thyroid carcinoma, medullary thyroid carcinoma, follicular thyroid carcinoma, anaplastic thyroid carcinoma), urinary system cancers (renal cell carcinoma, Wilms' tumor, urothelial carcinoma), male and female reproductive organ cancers (e.g., choriocarcinoma, seminoma, embryonal carcinoma, cervical cancer, testicular tumor, prostate cancer, ovarian cancers), lung cancers (e.g., lung adenocarcinoma, small cell lung carcinoma, lung squamous cell carcinoma, lung large cell carcinoma, papillary adenocarcinomas, bronchogenic carcinoma), epithelial carcinoma, central or peripheral neural system cancers (e.g., glioma, astrocytomoma, pinealoma, hemangioblastoma, acoustic neuroma, oligodendroglioma, meningioma, neuroblastoma, and retinoblastoma), mesothelioma, or other undifferentiated or poorly differentiated cancers.

In some embodiments, GBL derivatives can be used for differentiation therapy of benign neoplasms such as adenomas, lipomas, leiomyomas, rhabdomyoma, fibroids, hemangiomas, meningiomas, neuromas, schwannomas, papillomas, hamartomas, chondromas, synovioma, osteoma, desmoid tumor, and hepatomas.

In some embodiments, GBL derivatives suitable for inducing differentiation of poorly differentiated neoplastic cells, e.g., cancer cells, include, but are not limited to:

HSL-C8, which has the following chemical structure:

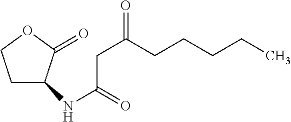 [SMILES: CCCCCC(=O)CC(=O)N[C@H]1CCOC1=O]

HSL-C12, which has the following chemical structure:

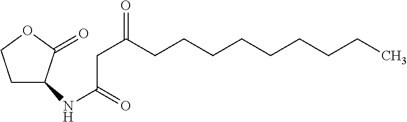 [SMILES: CCCCCCCCCC(=O)CC(=O)N[C@H]1CCOC1=O]

HSL-A, which has the following chemical structure:

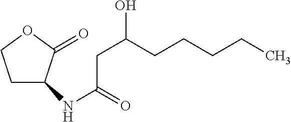 [SMILES: CCCCCC(O)CC(=O)N[C@H]1CCOC1=O]

HSL-B, which has the following chemical structure:

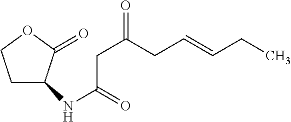 [SMILES: CC/C=C/CC(=O)CC(=O)N[C@H]1CCOC1=O]

HSL-C, which has the following chemical structure:

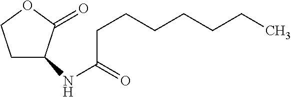 [SMILES: CCCCCCCC(=O)N[C@H]1CCOC1=O]

A-factor, which has the following chemical structure:

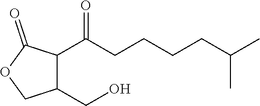 [SMILES: CC(C)CCCCC(=O)C1C(=O)OCC1CO]

GBL, which has the following chemical structure:

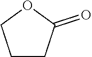

Here, we have demonstrated that GBL derivatives HSL-C8, HSL-C12, A-factor, and GBL can induce cell differentiation in neoplastic cells, e.g., cancer cells, when compared to untreated cells. In particular, we show that HSL-C8, HSL-C12, A-factor, and GBL can induced terminal differentiation in the poorly differentiated HL60 cell line of promyelocytic origin.

HSL-C8, HSL-C12, A-factor, and GBL also induce terminal differentiation in a KG1a cell line with poorly differentiated cells of myelocytic origin. By using gene expression analysis, microscopy, and flow cytometric analysis to study the differentiation inducing effects of GBL derivatives, it is found that HSL-C8, HSL-C12, A-factor, and GBL induce differentiation-related genes and CD markers, and change the morphology of undifferentiated cancer cells to differentiated cell lineages at non-toxic concentrations determined by cell viability assays and evaluation of apoptosis/necrosis. Minimal toxicity, and the ability of HSL-C8, HSL-C12, A-factor, and GBL to induce malignant reversion of cancer cells through the induction of differentiation, supports the use of GBL derivatives for differentiation therapy of cancers.

In some embodiments, GBL derivatives, including HSL-C8, HSL-C12, A-factor, and GBL, may be used to modulate the cellular differentiation of neoplastic cells by contacting the neoplastic cells with a subapoptotic concentration of GBL derivative. In some embodiments, GBL derivatives may be used for differentiation therapy of a neoplasm by contacting neoplastic cells of a patient with a subapoptotic concentration of a GBL derivative.

In some embodiments, the neoplasm is a cancer. In some embodiments cancers suitable for treatment in accordance with embodiments of the invention include solid or liquid tumors. In some embodiments cancers suitable for treatment in accordance with embodiments of the invention include blood and lymphatic system cancers (e.g., acute lymphoblastic leukemia, chronic lymphoblastic leukemia, acute myeloid leukemia, acute promyelocytic leukemia, chronic myeloid leukemia, Hodgkin lymphoma, non-hodgkin lymphoma, diffuse large B-cell lymphoma, lymphangiosarcoma, lymphangioendotheliosarcoma, endotheliosarcoma), musculoskeletal cancers (e.g., synovial carcinoma, chondrosarcoma, Ewing's tumor, leiomyosarcoma, myosarcoma, rhabdomyosarcoma, liposarcoma, fibrosarcoma), gastrointestinal cancers (e.g., esophageal carcinoma, pancreatic adenocarcinoma, stomach adenocarcinoma, colon adenocarcinoma, bile duct carcinoma), breast cancers (e.g., ductal carcinoma, lobular carcinoma, tubular/cribriform carcinoma, mucinous carcinoma, medullary carcinoma, papillary carcinoma), skin cancers (e.g., squamous cell carcinoma, basal cell carcinoma, melanoma, sweat gland carcinoma, sebaceous gland carcinoma), thyroid cancers (papillary thyroid carcinoma, medullary thyroid carcinoma, follicular thyroid carcinoma, anaplastic thyroid carcinoma), urinary system cancers (renal cell carcinoma, Wilms' tumor, urothelial carcinoma), male and female reproductive organ cancers (e.g., choriocarcinoma, seminoma, embryonal carcinoma, cervical cancer, testicular tumor, prostate cancer, ovarian cancers), lung cancers (e.g., lung adenocarcinoma, small cell lung carcinoma, lung squamous cell carcinoma, lung large cell carcinoma, papillary adenocarcinomas, bronchogenic carcinoma), epithelial carcinoma, central or peripheral neural system cancers (e.g., glioma, astrocytomoma, pinealoma, hemangioblastoma, acoustic neuroma, oligodendroglioma, meningioma, neuroblastoma, and retinoblastoma), mesothelioma.

In some embodiments, the neoplasm is a benign neoplasm. In some embodiments benign neoplasms suitable for treatment in accordance with embodiments of the invention include solid or liquid tumors. In some embodiments benign neoplasms suitable for treatment in accordance with embodiments of the invention include adenomas, lipomas, leiomyomas, rhabdomyoma, fibroids, hemangiomas, meningiomas, neuromas, schwannomas, papillomas, hamartomas, chondromas, synovioma, osteoma, desmoid tumor, and hepatomas.

In some embodiments, more than one type of neoplastic cell is contacted with a subapoptotic concentration of GBL derivative.

In some embodiments, a single type of neoplastic cell or a mixture of at least two types of neoplastic cells may be cultivated in vitro. In some embodiments, the neoplastic cells are implanted ex-vivo. In some embodiments, the targeted neoplastic cells are present in vivo in a mammal such as humans.

In some embodiments HSLs include 4 to 14 carbons on their acyl chain. In some embodiments, HSL acyl chains comprise at least one functional group, e.g., oxo or hydroxyl functional groups.

In some embodiments, GBL derivatives are used alone. In some embodiments, a mixture of at least two GBL derivatives are used to induce differentiation of a neoplastic cell.

In some embodiments GBL derivatives are produced naturally. In some embodiments GBL derivatives are produced synthetically or semi-synthetically. In some embodiments GBL derivatives are produced by an organism and the produced GBL derivatives induce differentiation through a direct or indirect contact of the organism with cancer cells.

In some embodiments, a GBL derivative may be administered orally, rectally, parenterally, intravenously, or topically. In some embodiments, a GBL derivative may be administered along with at least one appropriate pharmaceutical carrier selected with regard to the intended route of administration according to standard pharmaceutical practices.

In some embodiments, a GBL derivative is formulated in the form of tablets, capsules, creams, lotions, or sterile solutions for induction of differentiation and differentiation therapy. In some embodiments, a GBL derivative can be loaded or coated inside or on a carrier for induction of differentiation and differentiation therapy. In some embodiments, a GBL derivative can be dissolved in hydrophobic or hydrophilic solvents for induction of differentiation and differentiation therapy.

In some embodiments, a GBL derivative can be coated or bound onto materials and compositions for in vivo or in vitro applications. Such materials include, but are not limited to, polymers, grafts, membranes, implants, fibers, beads, and cell culture containers and devices for induction of differentiation and differentiation therapy.

In some embodiments, contacting a neoplastic cell with a subapoptotic concentration of GBL derivative equal to or less than 500 μM induces differentiation of the neoplastic cell. In some embodiments, contacting a neoplastic cell with a subapoptotic concentration of GBL derivative equal to or less than 400 μM induces differentiation of the neoplastic cell. In some embodiments, contacting a neoplastic cell with a subapoptotic concentration of GBL derivative equal to or less than 300 μM induces differentiation of the neoplastic cell. In some embodiments, contacting a neoplastic cell with a subapoptotic concentration of GBL derivative equal to or less than 250 μM induces differentiation of the neoplastic cell. In some embodiments, contacting a neoplastic cell with a subapoptotic concentration of GBL derivative equal to or less than 200 μM induces differentiation of the neoplastic cell. In some embodiments, contacting a neoplastic cell with a subapoptotic concentration of GBL derivative equal to or less than 160 μM induces differentiation of the neoplastic cell. In some embodiments, contacting a neoplastic cell with a subapoptotic concentration of GBL derivative equal to or less than 150 μM induces differentiation of the neoplastic cell. In some embodiments, contacting a neoplastic cell with a subapoptotic concentration of GBL derivative equal to or less than 120 μM induces differentiation of the neoplastic cell. In some embodiments, contacting a neoplastic cell with a subapoptotic concentration of GBL derivative equal to or less than 100 μM induces differentiation of the neoplastic cell. In some embodiments, contacting a neoplastic cell with a subapoptotic concentration of GBL derivative equal to or less than 80 μM induces differentiation of the neoplastic cell. In some embodiments, contacting a neoplastic cell with a subapoptotic concentration of GBL derivative equal to or less than 50 μM induces differentiation of the neoplastic cell. In some embodiments, contacting a neoplastic cell with a subapoptotic concentration of GBL derivative equal to or less than 40 μM induces differentiation of the neoplastic cell. In some embodiments, contacting a neoplastic cell with a subapoptotic concentration of GBL derivative equal to or less than 25 μM induces differentiation of the neoplastic cell.

In some embodiments, the GBL derivative is selected from the group consisting of HSL-C8, HSL-C12, HSL-A, HSL-B, HSL-C, A-factor, GBL, and combinations thereof.

In some embodiments, induced differentiation is marked by increased expression of FLT3, PU1, CEBPA, and combinations thereof.

In some embodiments, induced differentiation is marked by an increase in the number of cells expressing CD3, CD20, CD24, CD33, CD16, CD56, and combinations thereof.

Protocols

Cell culture methods and treatments. HL60 and KG1a cell lines were cultivated in RPMI 1640 (Sigma-USA) culture medium supplemented with 1% penicillin/streptomycin (Gibco-USA), 10% and 20% fetal bovine serum (Gibco-USA) for HL60 and KG1a cell lines, respectively, and 0.5% Glutamax (Gibco-USA) for HL60 cell line. The cells were incubated at 37° C., 5% CO₂, and 90% humidity. The stock solution (Mol/L) of HSL-C8, HSL-C12, and A-factor were prepared in 0.1% (v/v) and 0.2% (v/v) and 0.2% (v/v) DMSO, respectively, as a vehicle and stored at −80° C. Different dilutions of HSL-C8, HSL-C12, A-factor, and GBL were prepared from the stock solution, and 41 μL (<4% of the total volume) of each were added to the wells containing 100 μL of cell suspension (10000 and 20000 HL60 and KG1a cells per well). 0.1% and 0.2% DMSO were used as the negative control for HSL-C8, HSL-C12, and A-factor for each test. Culture medium were used as the negative control for GBL.

Cell viability assay. The cell viability was evaluated via (MTS) assay. HL60, KG1a, and PBMC cells were treated with HSL-C8, HSL-C12, A-factor, and GBL in 96-well plates for 24, 48, and 72 hours. Thereafter, 204, of MTS/PMS solution were added to each well and the cells were incubated for 90 minutes. The optical density (OD) of samples was measured via spectrophotometer (EPOCH, BioTek, USA) at 490 nm and the viability of cells was calculated via the following formula:

Cell viability (%)=[ODₛₐₘₚₗₑ/OD_{control}]×100

Flow cytometry. HL60 and KG1a cells treated with HSL-C8, HSL-C12, A-factor, and GBL were stained with Annexin-V-FITC/PI for apoptosis/necrosis analysis and PI for cell cycle analysis. In addition, treated cells were incubated with antibody-dye conjugate against CD3, CD20, CD16, CD56, CD14, and CD33 for surface antigen analysis. Thereafter the samples were analyzed by flow cytometer (Attune NxT, Life Technologies-Thermo Fisher Scientific, USA). Treatment with 5 μL H₂O₂ and 1 μM ATRA were used as the positive controls for apoptosis and necrosis and surface antigen analyses, respectively. Data were analyzed with FlowJo software.

Wright-Giemsa staining. HL60 and KG1a cells treated HSL-C8 and HSL-C12 were concentrated and then fixed on the slides with absolute ethanol prior to Wright-Giemsa staining. After staining for four minutes the 1 mL of tap water was added to the slides. After four minutes, they were washed with tap water, air-dried, and evaluated with a light microscopy.

RNA isolation and qRT-PCR. The RNA of HL60 and KG1a cells treated with HSL-C8, HSL-C12, A-factor, and GBL were isolated manually via TRIzol, Chloroform, Ethanol, and Isopropanol. The concentration of extracted RNA was measured via spectrophotometer (EPOCH, BioTek, USA) at 260 nm. The quality of isolated RNA was further evaluated via agarose gel electrophoresis indicating two distinctive bands. Isolated RNA was then converted to cDNA via a commercial kit according to the manufacturer's instructions. The primers were designed with AlleleID software and Primer3 algorithm and then double-checked via the BLAST algorithm. cDNA samples were then prepared for analysis using a commercial kit and analyzed with qRT-PCR machine (Applied Biosystems-Thermo Fisher Scientific, USA).

Statistical analysis. The statistical analyses were done using GraphPad Prism 6.01 (GraphPad Software, San Diego, CA, USA) and Excel (Microsoft, USA). All experiments were done with at least three replicates. The statistical significance was set at p-value<0.05.

### Example 1: HSL-C8 and HSL-C12 Induce Differentiation of Myelocytic and Promyelocytic Leukemia Cells

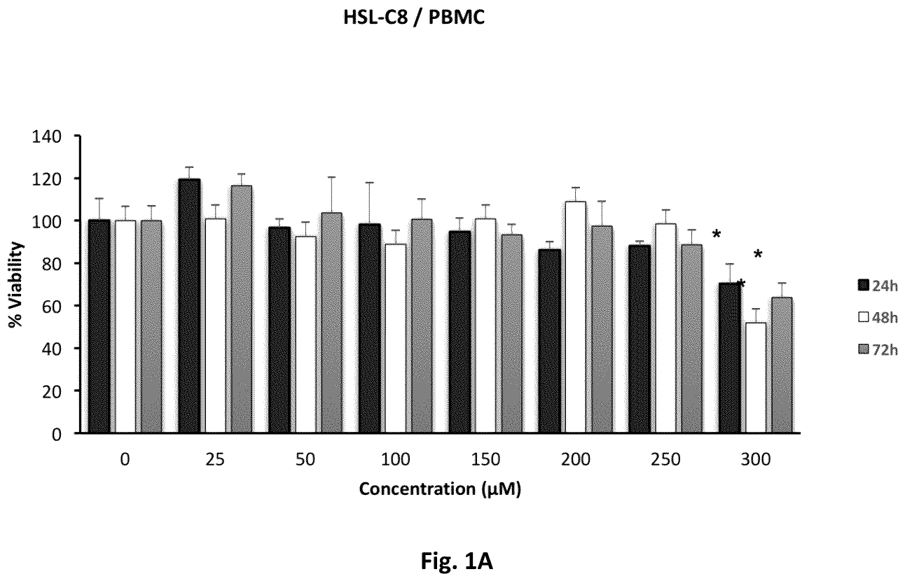

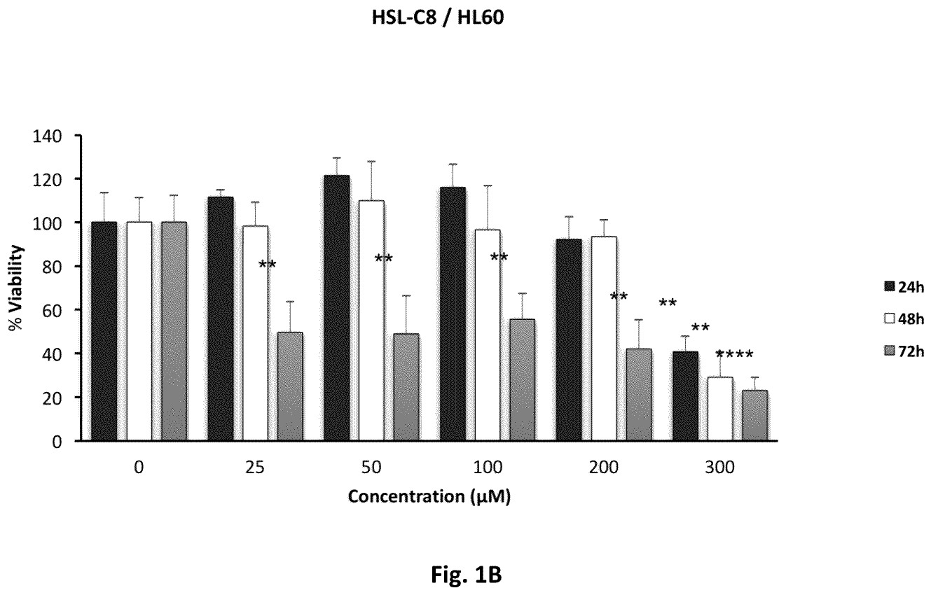

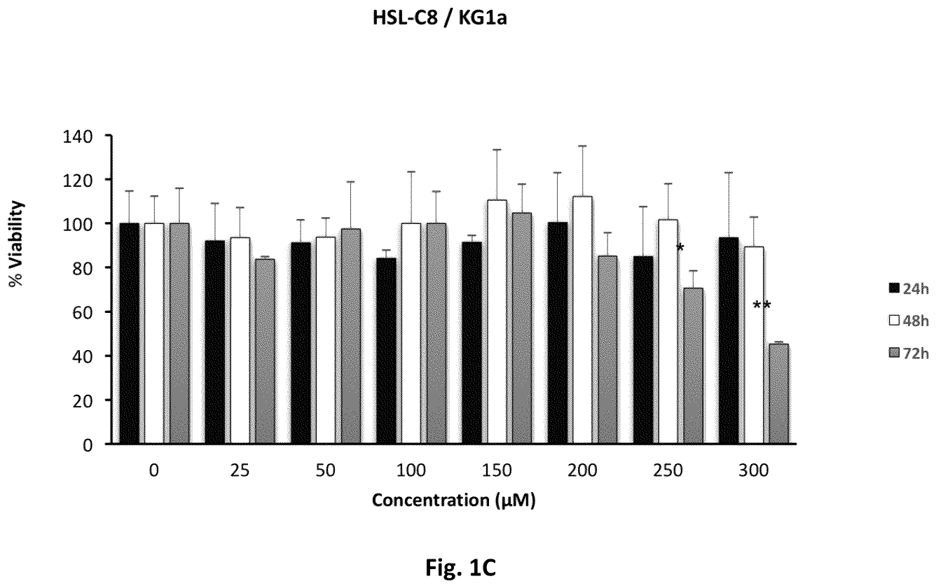

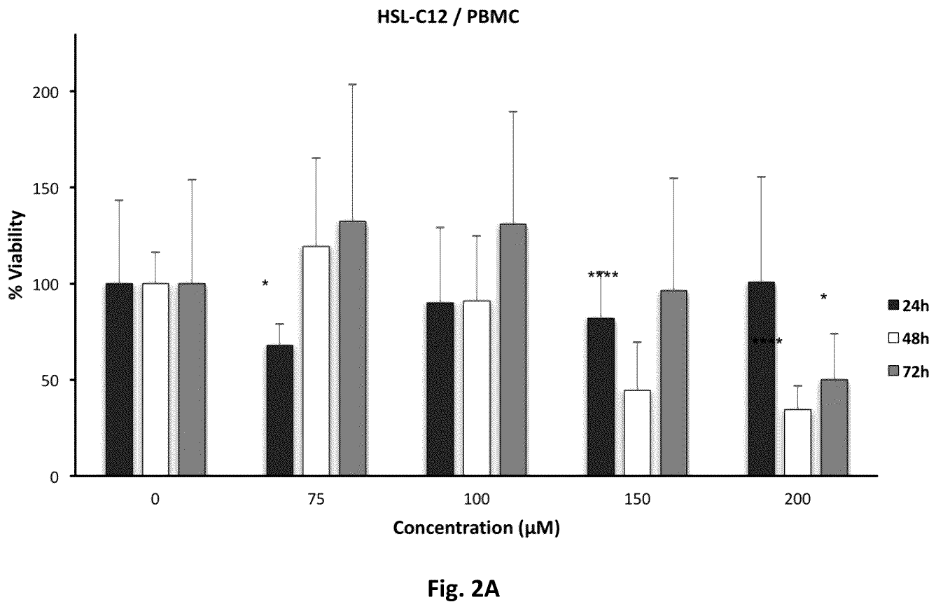

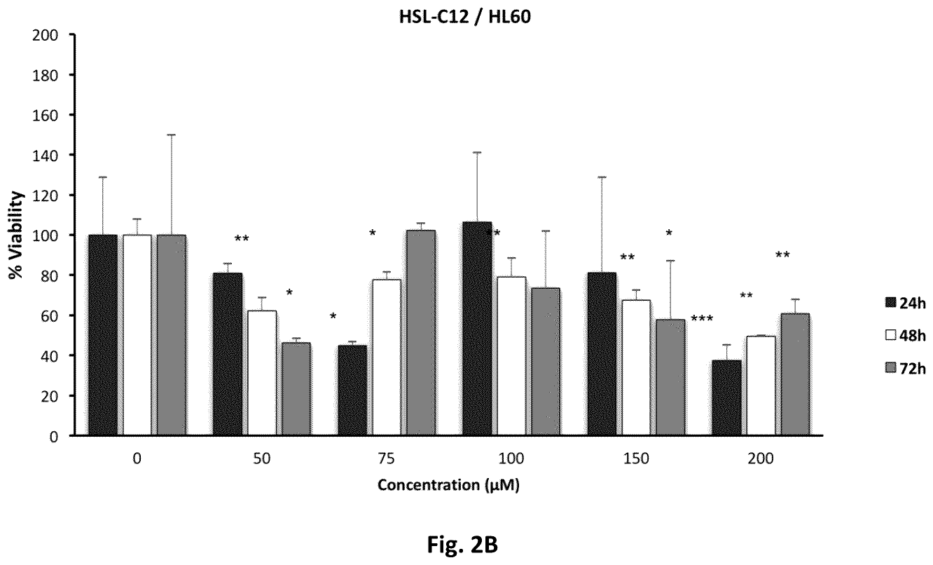

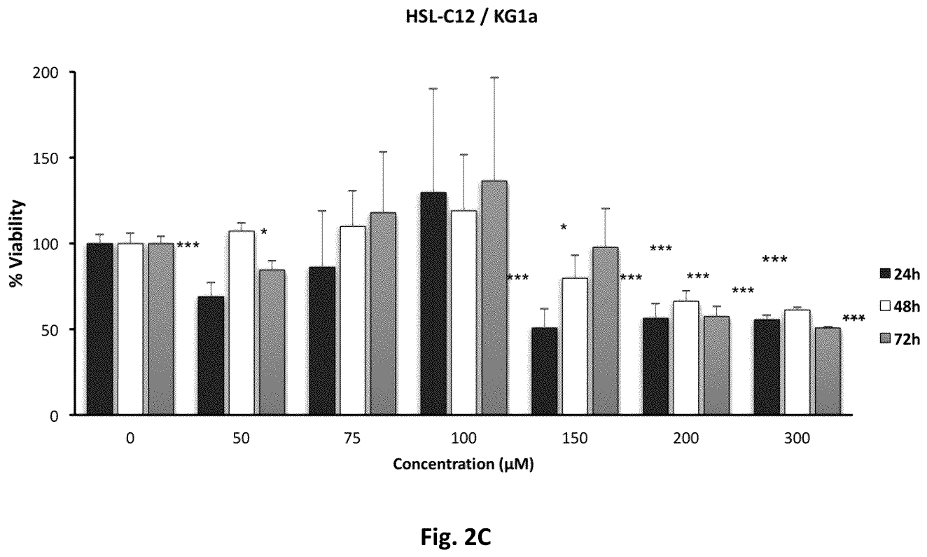

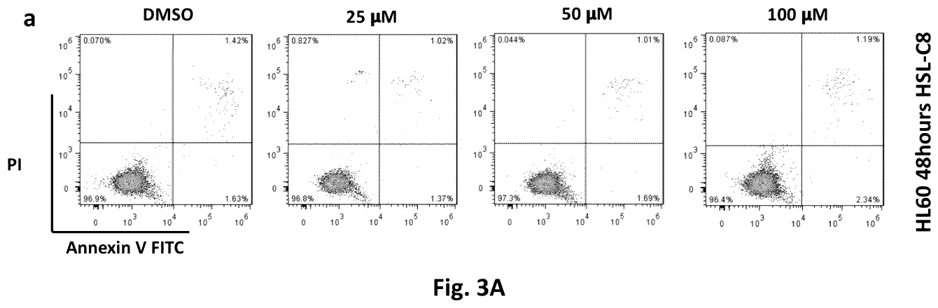

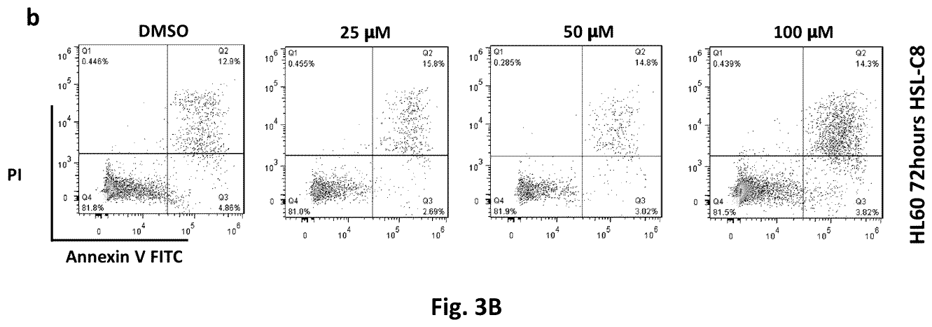

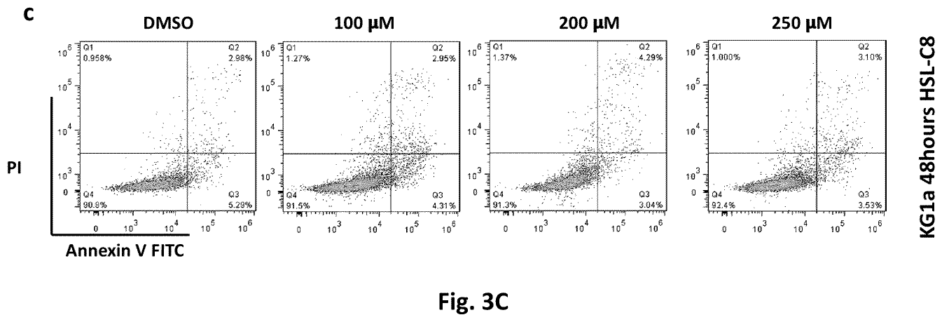

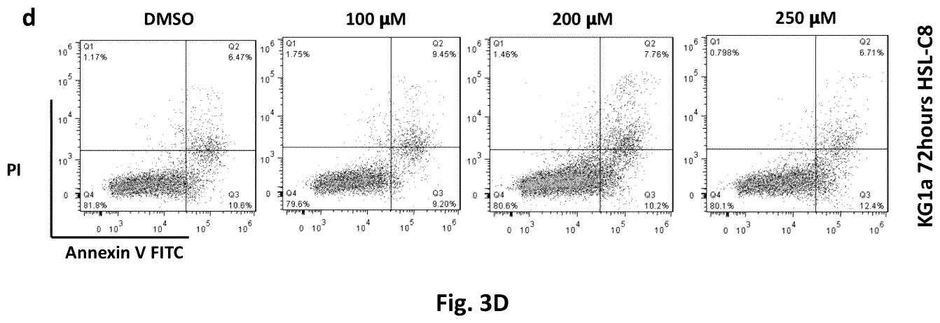

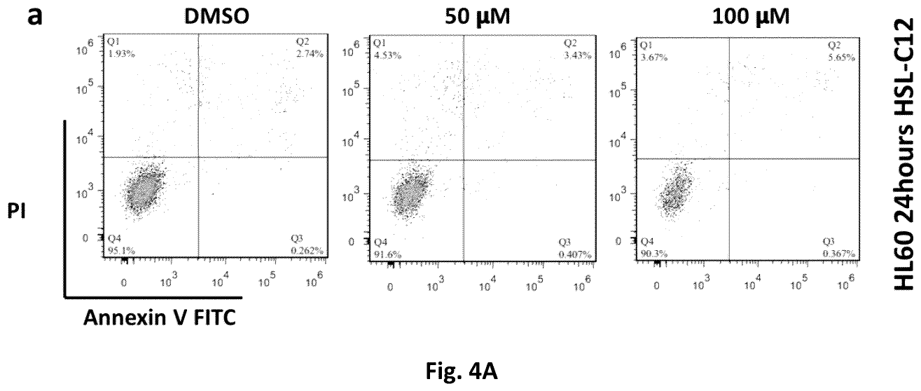

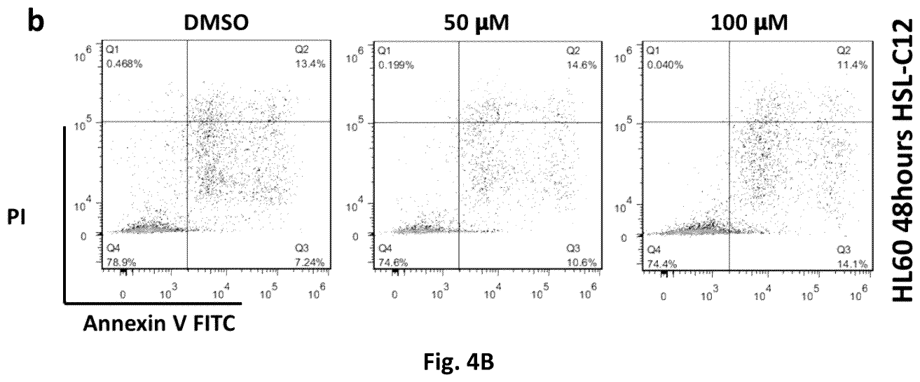

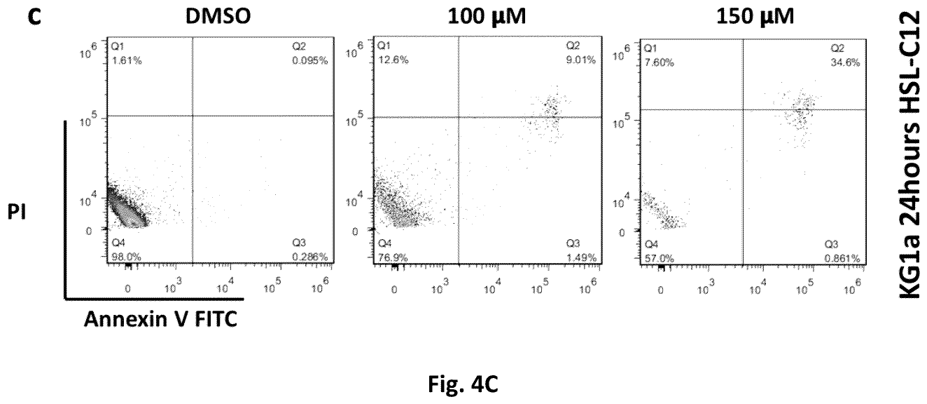

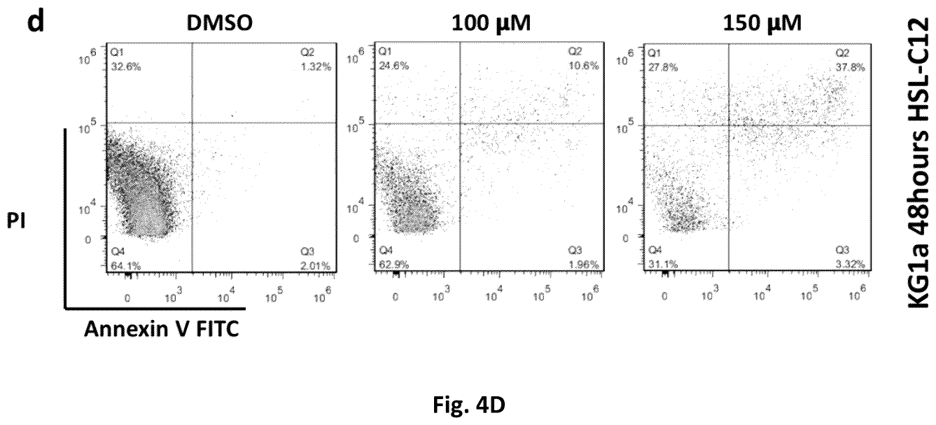

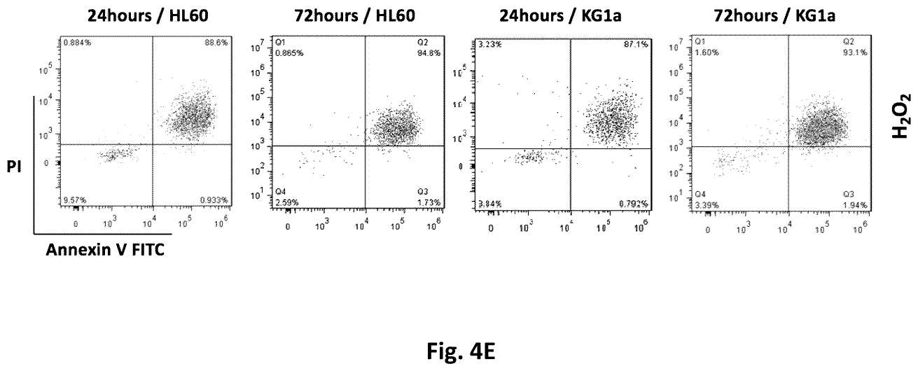

HSL-C8 and HSL-C12 induce differentiation of poorly differentiated cancer cells, including HL60 and KG1a cell lines, of promyelocytic and myelocytic origin, respectively. In order to evaluate the minimal toxicity of HSL-C8 and HSL-C12, cell viability analysis via calorimetry and apoptosis/necrosis analysis via flow cytometry was undertaken. HSL-C8 and HSL-C12 demonstrated minimal toxicity in both HL60 and KG1a cell lines at a concentration of at least below 150 μM, as shown in FIGS. 1A-C, FIGS. 2A-C, FIGS. 3A-D, and FIGS. 4A-D.

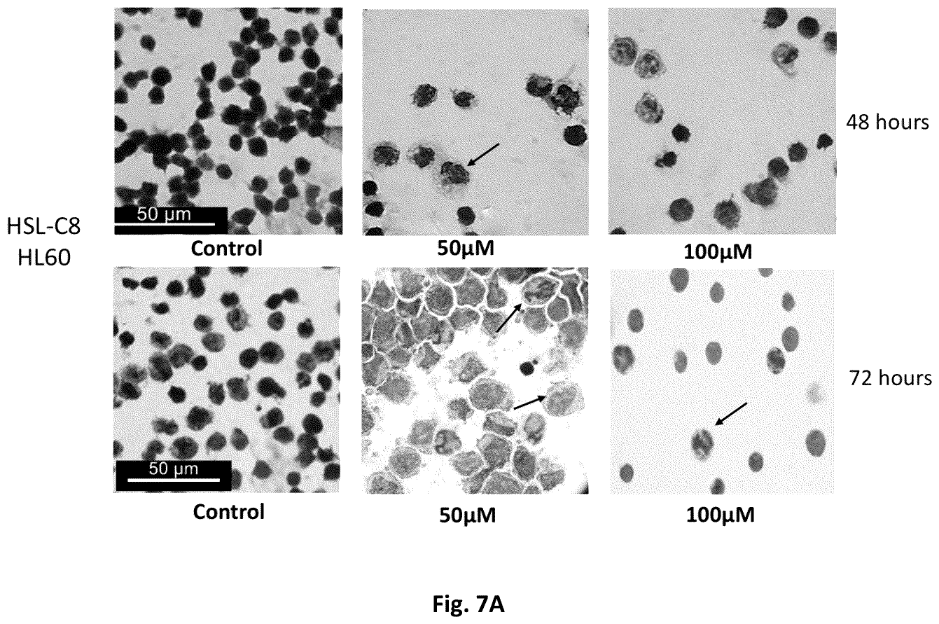

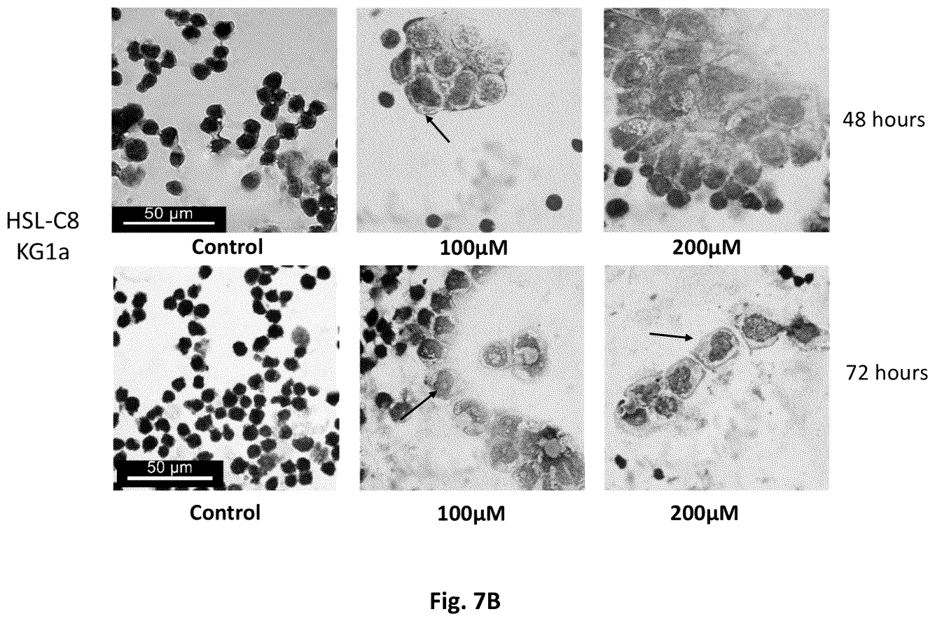

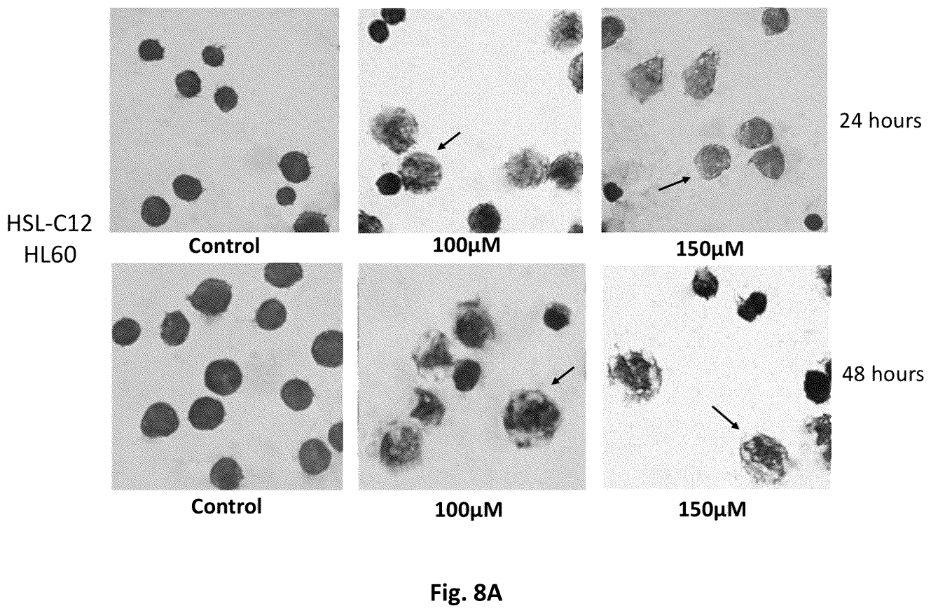

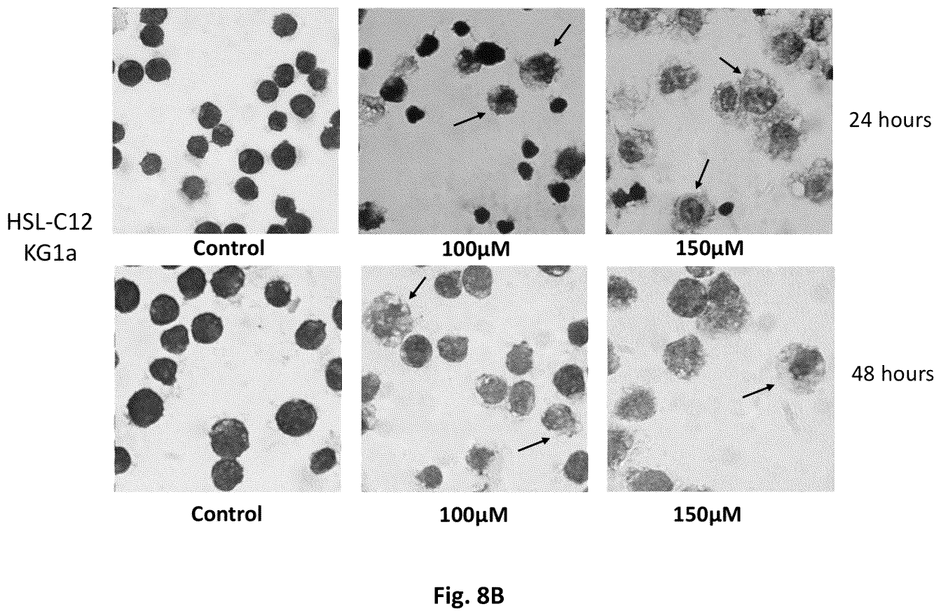

Furthermore, HSL-C8 and HSL-C12 induced terminal cellular differentiation in HL60 and KG1a cell lines, confirmed by gene expression, flow cytometric, and morphological analyses. As shown in FIGS. 7A-B and FIGS. 8A-C, contacting HL60 cells with 50 μM and 100 μM of HSL-C8, KG1a cells with 100 μM and 200 μM of HSL-C8, HL60 cells with 100 μM and 150 μM of HSL-C12, and KG1a cells with 100 μM and 150 μM of HSL-C12, changes the morphology of HL60 and KG1a cells to neutrophil and macrophage-like cells, with lobulated nuclei and expanded cytoplasm. These characteristics demonstrate the differentiation of HL60 and KG1a cells to differentiated myelocytic lineages.

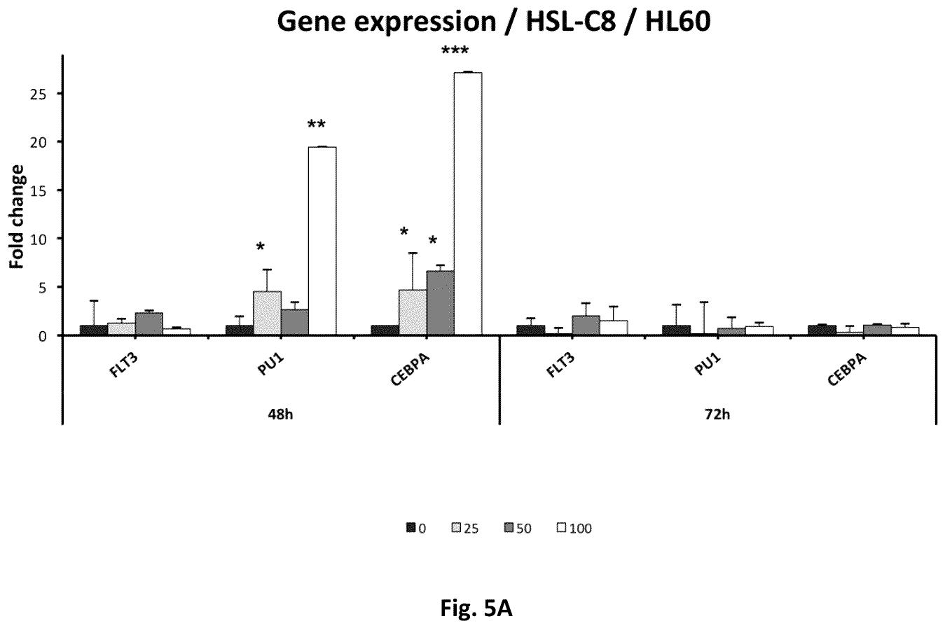

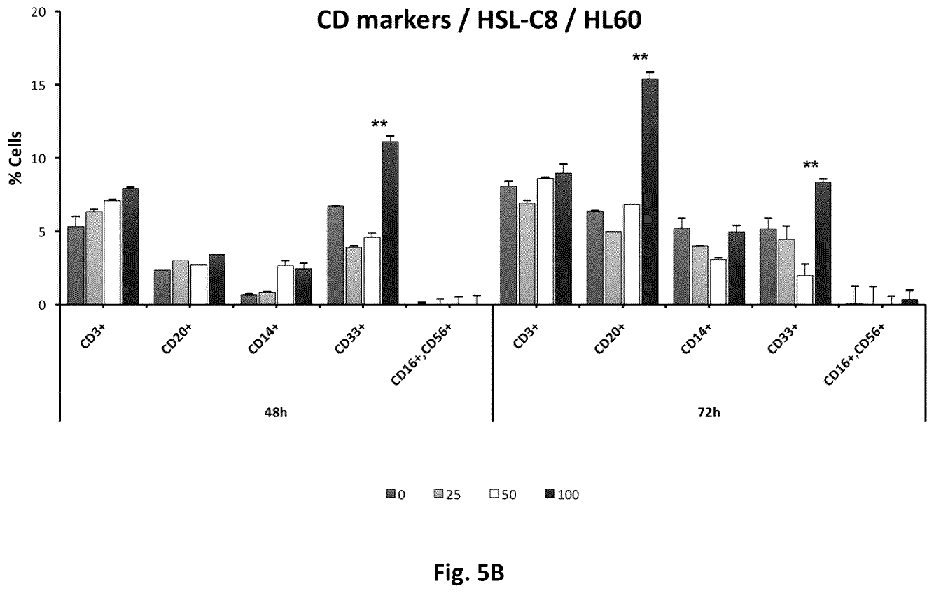

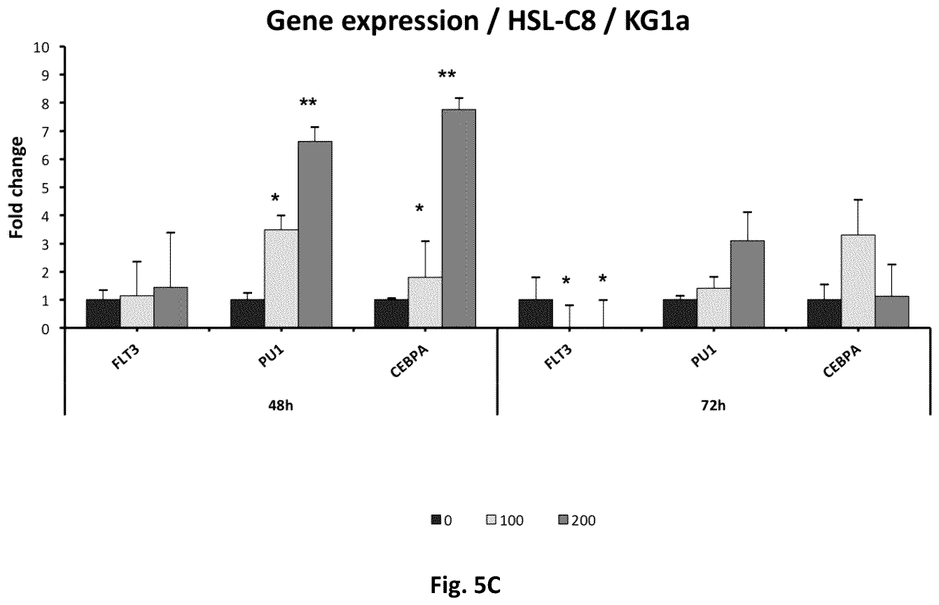

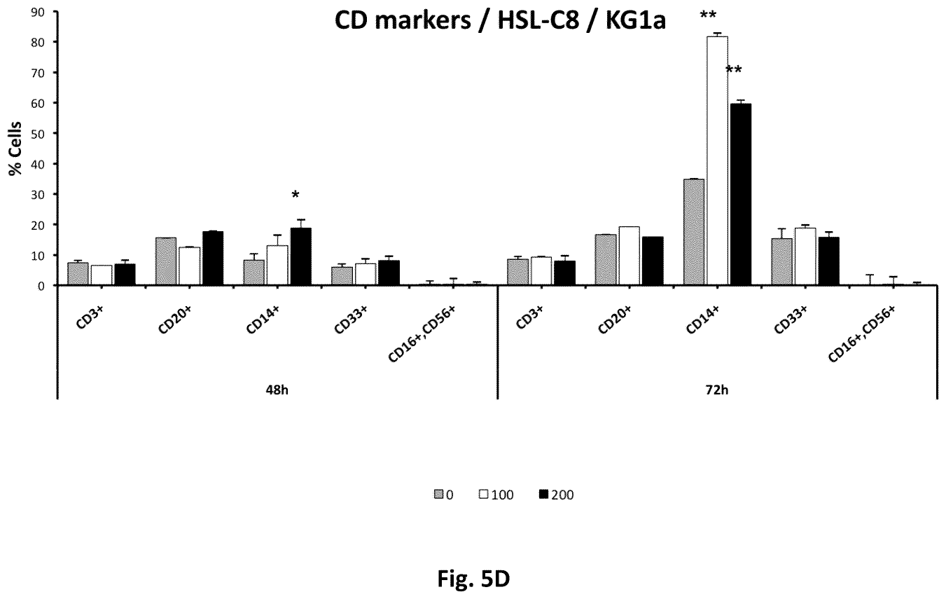

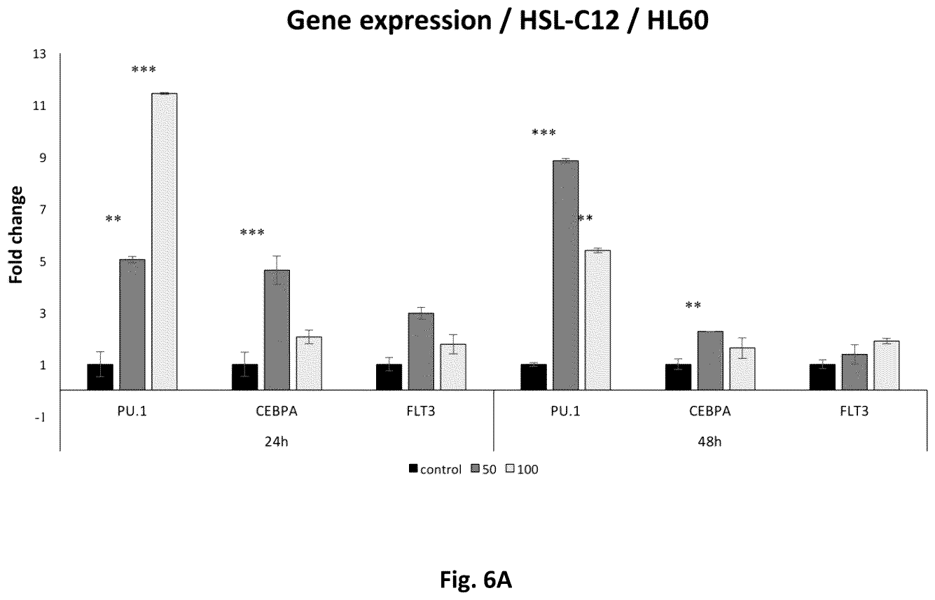

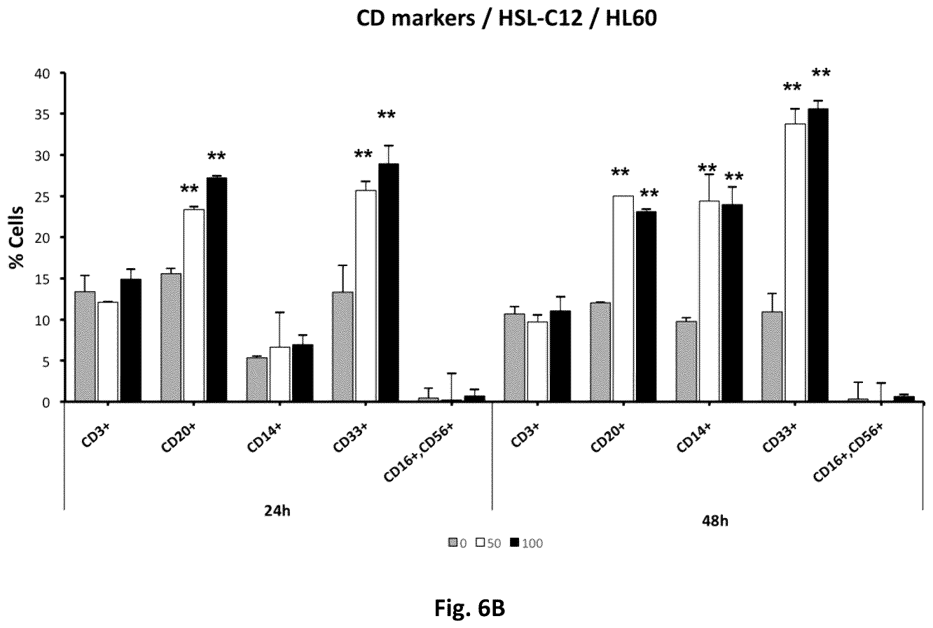

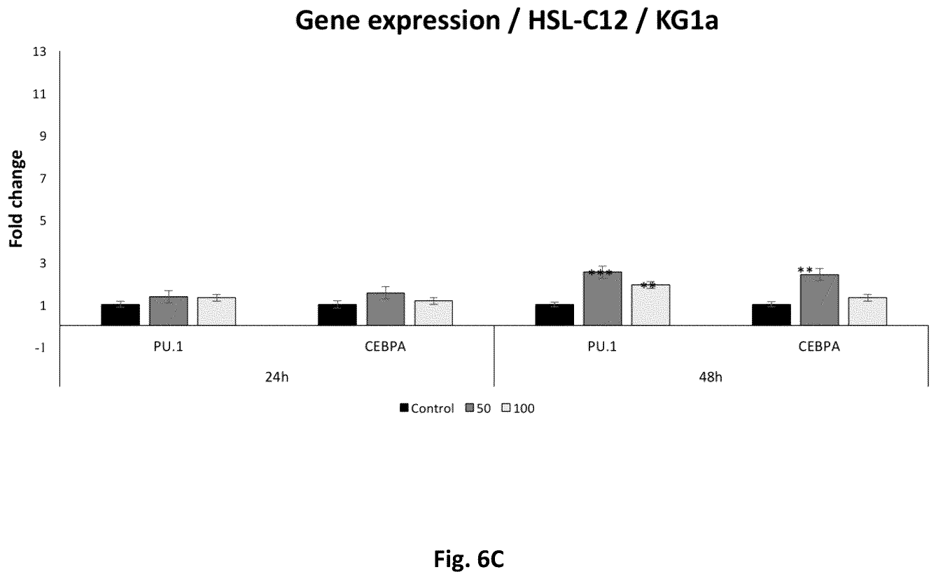

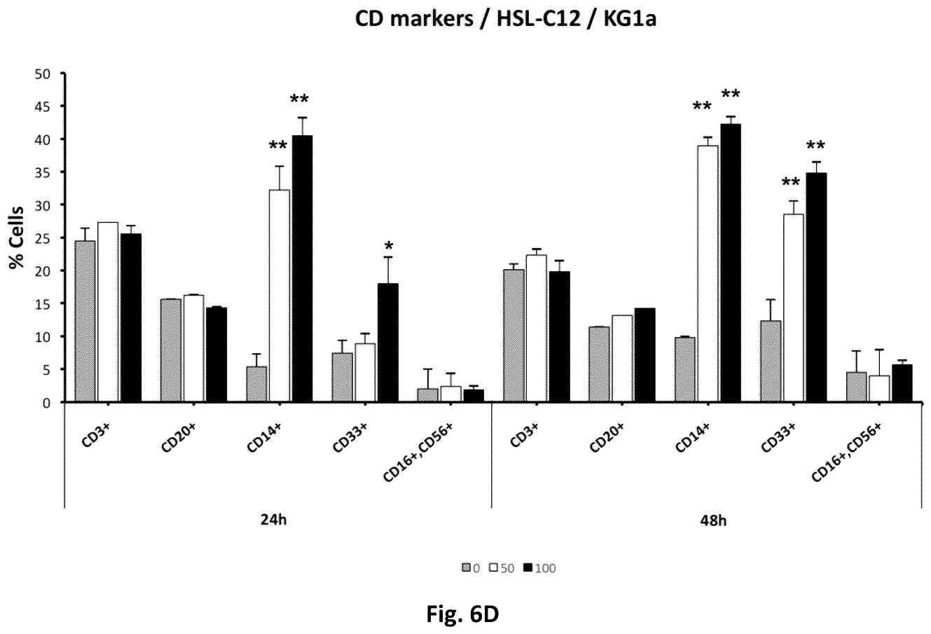

The expression of FLT3, PU1, and CEBPA genes and CD3, CD20, CD24, CD33, CD16, and CD56 surface markers (as in differentiated cell from myeloid lineage such as monocytes and neutrophils) shown in FIGS. 5A-D and FIGS. 6A-D confirms the terminal cellular differentiation of HL60 and KG1a cells. Moreover, HSL-C8 and HSL-C12, at the given concentrations, do not decrease the cellular viability of treated cancer cells and normal cells (PBMC) significantly, as shown in FIGS. 1A-C and FIGS. 2A-C. Therefore, the use of GBL derivatives, including HSL-C8 and HSL-C12, may be effectively utilized in differentiation therapy of cancers, targeting cancer cells specifically, and sparing normal (non-cancer) cells, leading to minimal toxicity and minimal side effects in the treated patient.

Moreover, FIGS. 3A-D and 4A-D demonstrate that HSL-C8 and HSL-C12 cause minimal to no apoptosis at concentrations of at least 100 μM or less. Upon a treatment with various concentrations of HSL-C8 and HSL-C12, the vast majority of HL60 and KG1a cells remained viable and non-apoptotic, demonstrating their minimal toxicity, consistent with the results of MTS analysis. These scatter plots of FIGS. 4A-D can be compared to the positive control results shown in FIG. 4E, obtained from the treatment of HL60 and KG1a cells with H₂O₂ as an apoptosis-inducing compound.

The lack of apoptosis in response to the treatment with HSL-C8 and HSL-C12 is a prerequisite for induction of differentiation in cancer cells and differentiation therapy of cancers. These results confirm that in contrast to previous toxic methods of treating cancer cells with HSLs, HSLs can be used at subapoptotic concentrations using a method that induces differentiation in cancer cells so as to treat cancers with minimal side effects.

### Example 2: A factor (2-isocapryloyl-3R-hydroxymethyl-gamma-butyrolactone) and GBL Induce Differentiation of Myelocytic and Promyelocytic Leukemia Cells

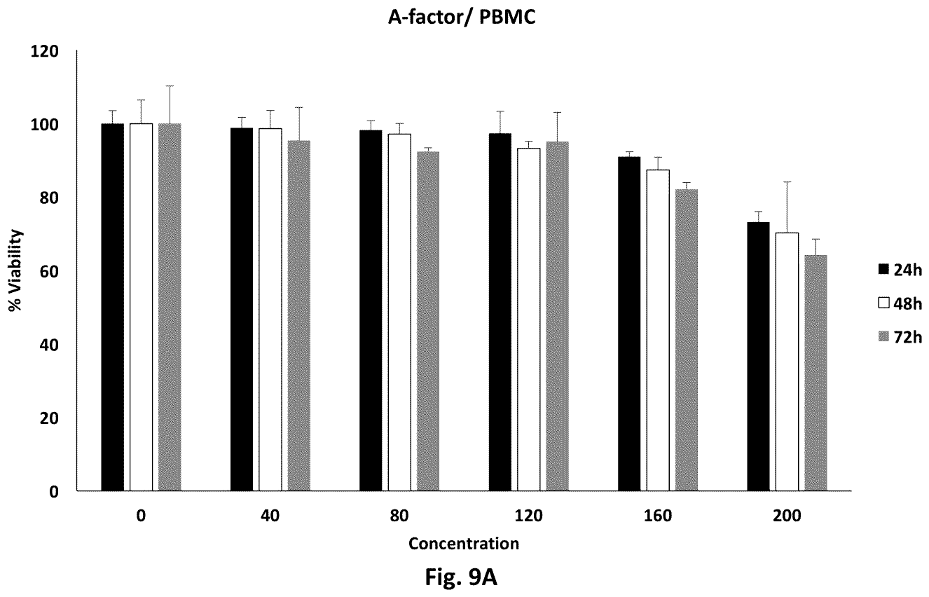

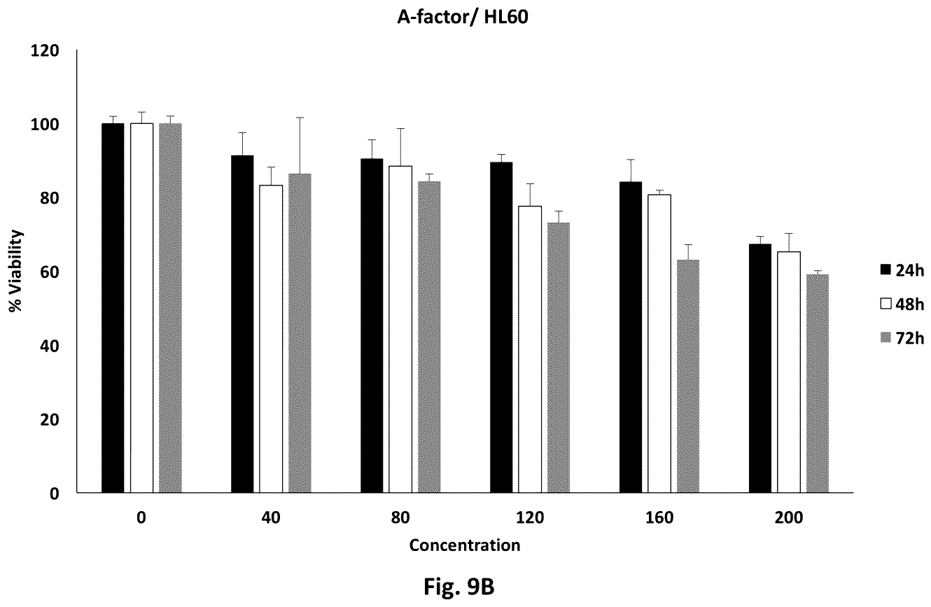

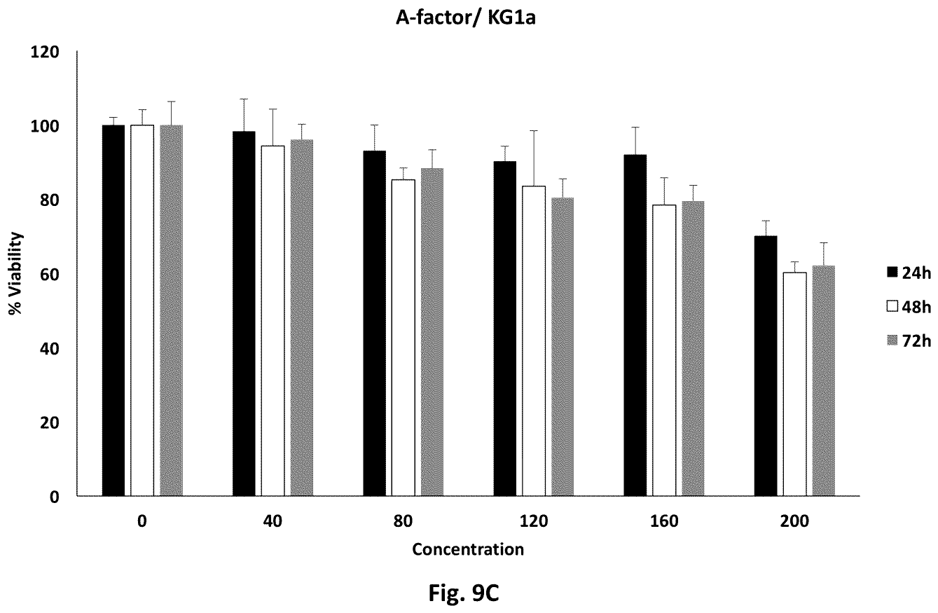

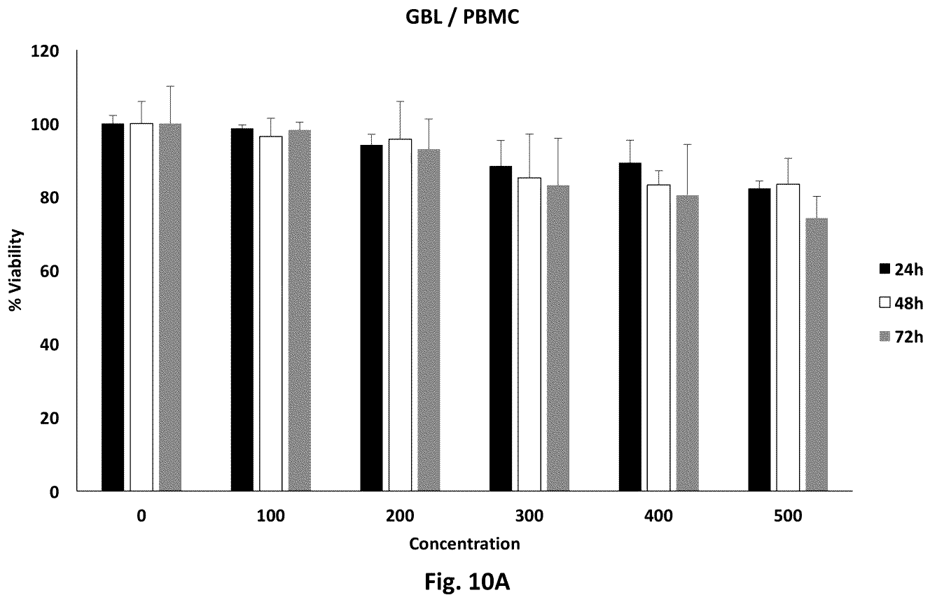

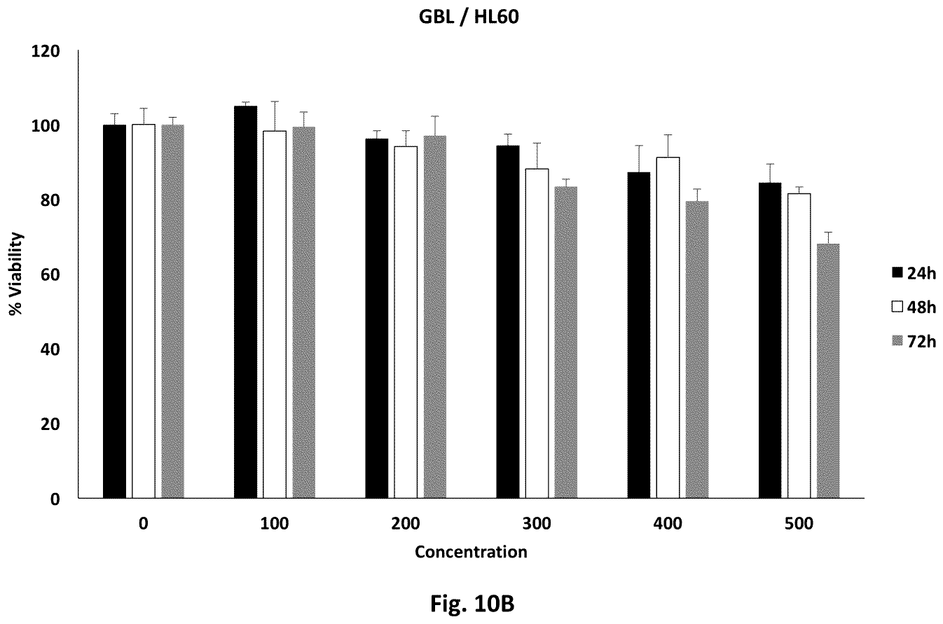

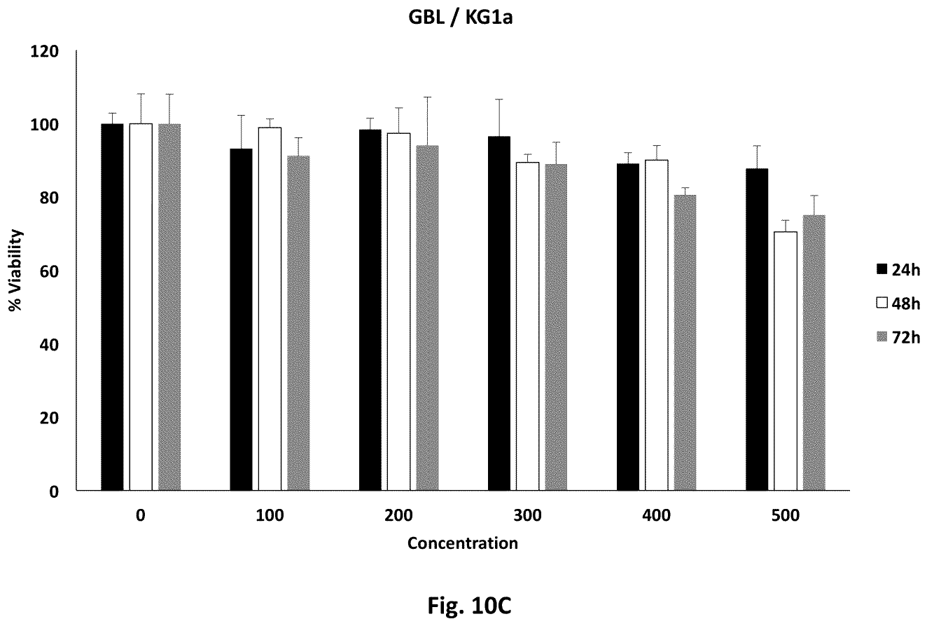

A-factor and GBL induce differentiation of poorly differentiated cancer cells, including HL60 and KG1a cell lines, of promyelocytic and myelocytic origin, respectively. In order to evaluate the minimal toxicity of A-factor and GBL, cell viability analysis via calorimetry and apoptosis/necrosis analysis via flow cytometry was undertaken. A-factor and GBL demonstrated minimal toxicity in both HL60 and KG1a cell lines at a concentration of induction of cellular differentiation, as shown in FIGS. 9B-C and FIGS. 10B-C.

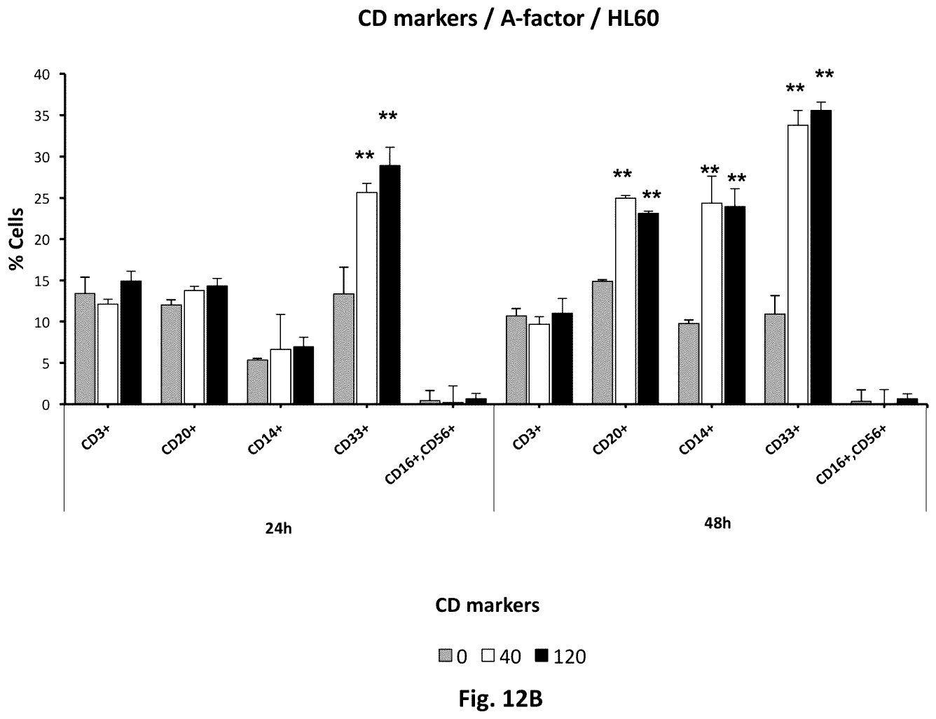

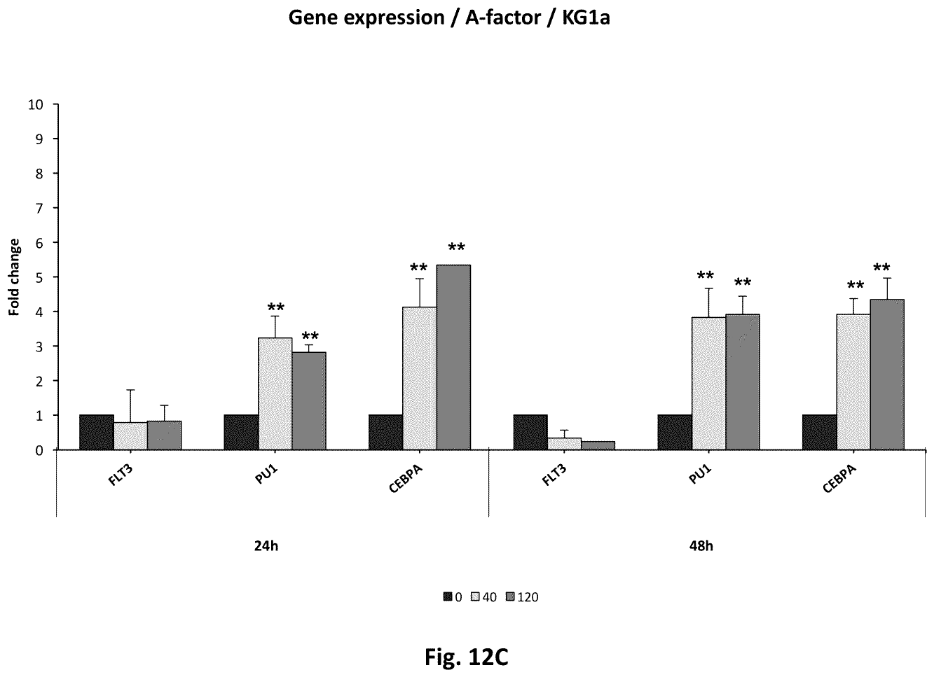

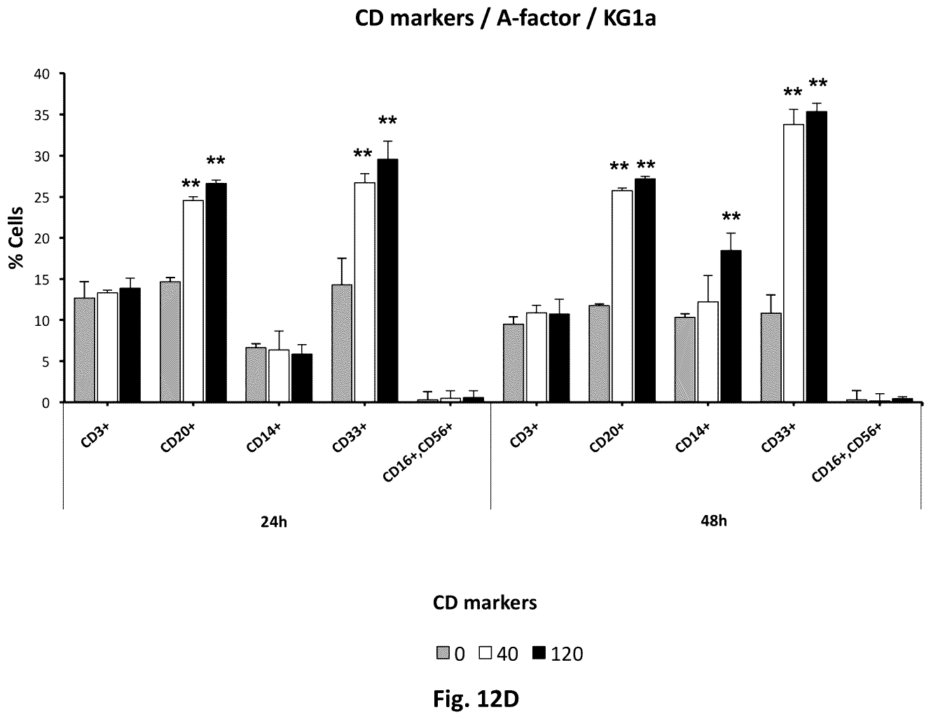

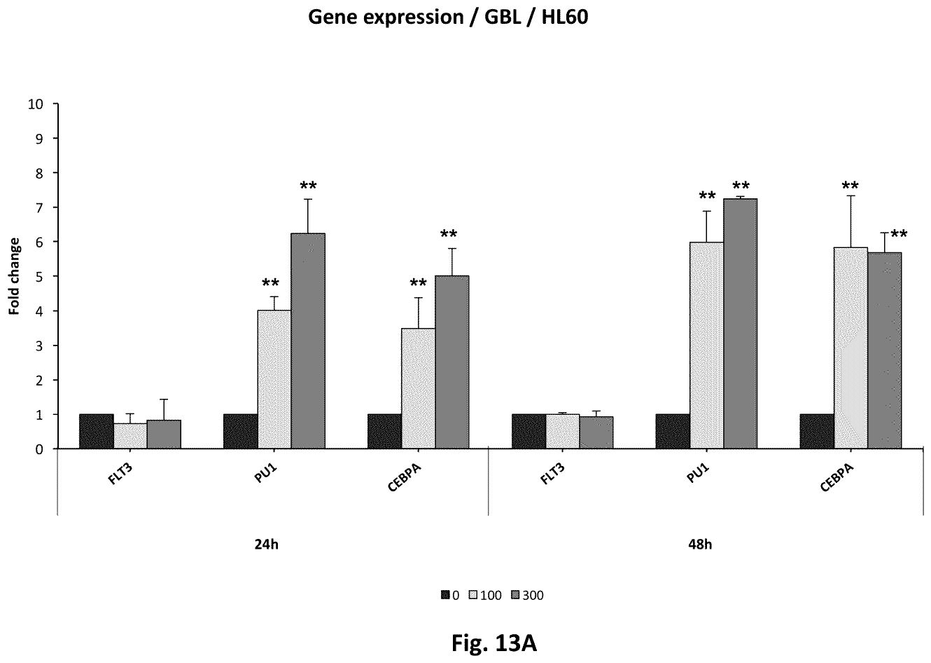

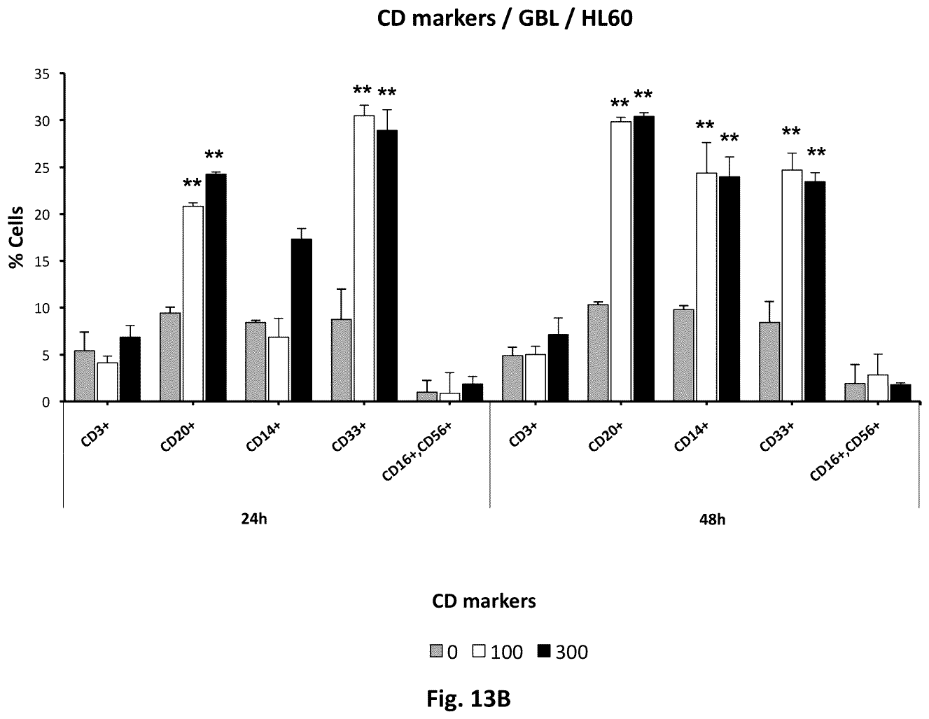

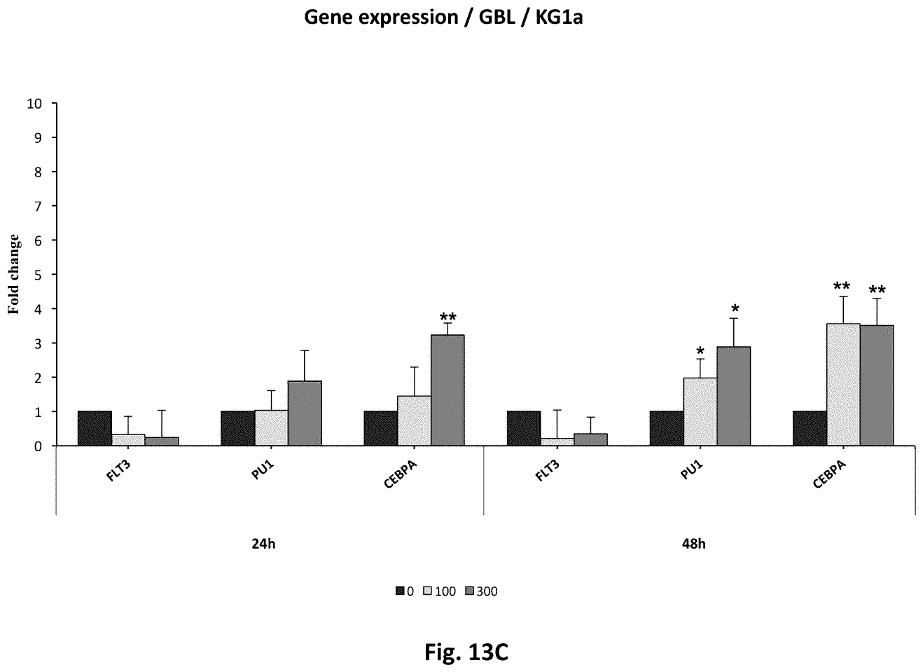

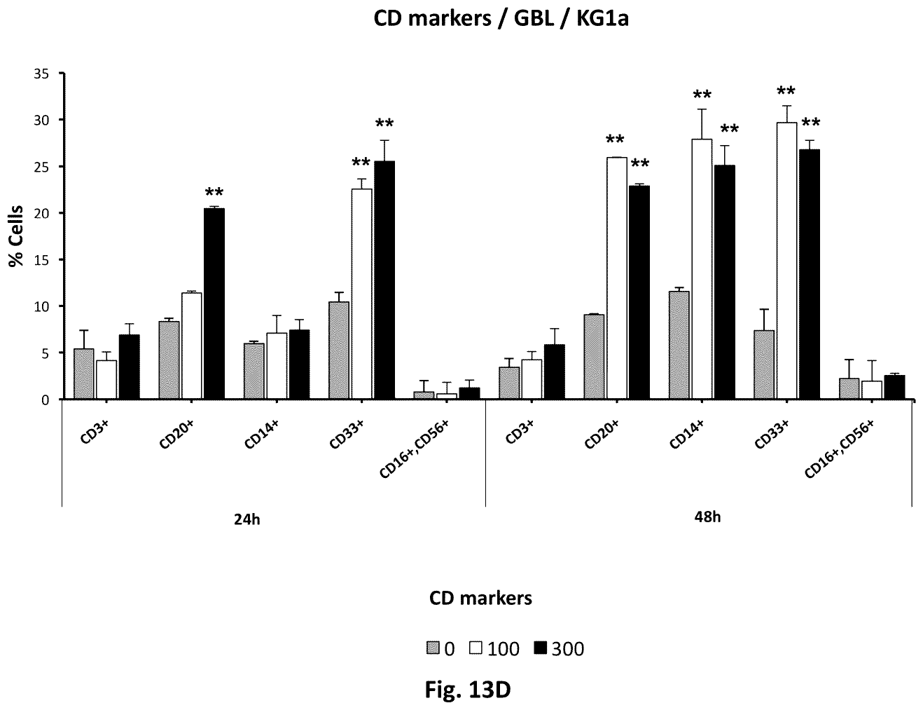

Furthermore, A-factor and GBL induced terminal cellular differentiation in HL60 and KG1a cell lines, confirmed by gene expression and flow cytometric analyses. The expression of FLT3, PU1, and CEBPA genes and CD3, CD20, CD24, CD33, CD16, and CD56 surface markers (as in differentiated cell from myeloid lineage such as monocytes and neutrophils), shown in FIGS. 12A-D and 13A-D, confirms the terminal cellular differentiation of HL60 and KG1a cells. Moreover, A-factor and GBL, at the given concentrations, do not decrease the cellular viability of treated cancer cells and normal cells (PBMC) significantly, as shown in FIGS. 9A and 10A. Therefore, the use of GBL derivatives, including A-factor and GBL, may be effectively utilized in differentiation therapy of cancers, targeting cancer cells specifically, and sparing normal (non-cancer) cells, leading to minimal toxicity and minimal side effects in the treated patient.

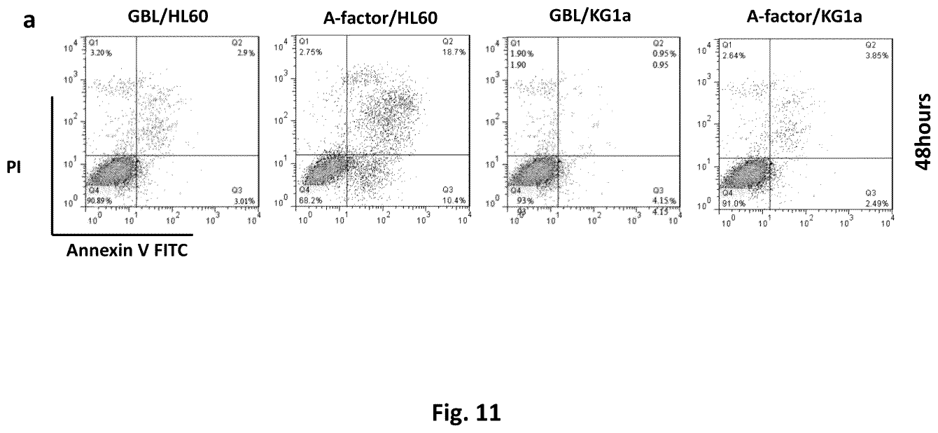

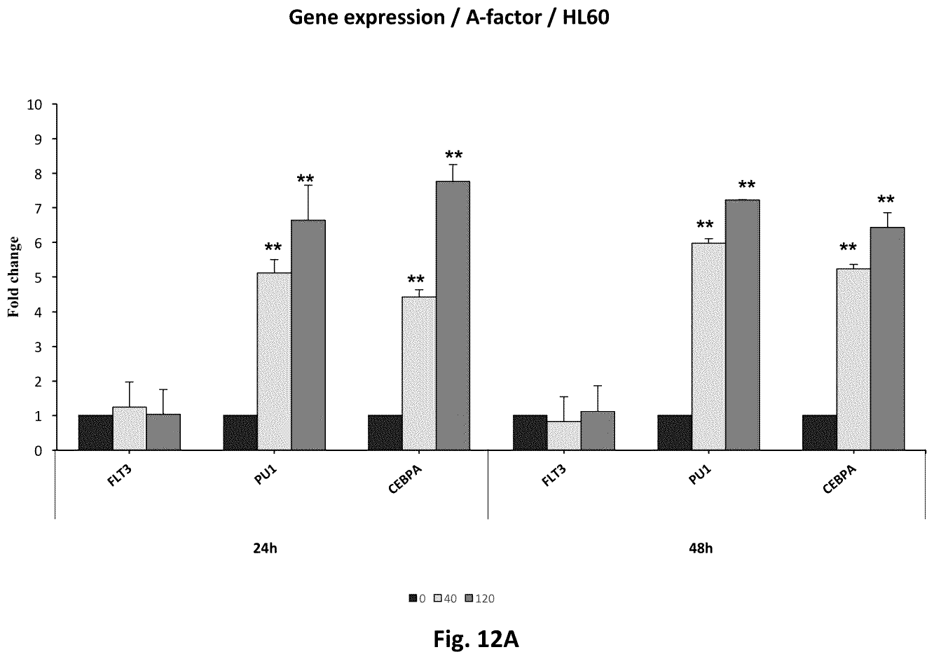

Moreover, FIG. 11 demonstrates that A-factor and GBL cause minimal apoptosis. Upon a treatment with various concentrations of A-factor and GBL, the vast majority of HL60 and KG1a cells remained viable and non-apoptotic, demonstrating their minimal toxicity, consistent with the results of MTS analysis. These scatter plots of FIG. 11 can be compared to the positive control results shown in FIG. 4E, obtained from the treatment of HL60 and KG1a cells with H₂O₂ as an apoptosis-inducing compound.

The lack of apoptosis in response to the treatment with A-factor and GBL is a prerequisite for induction of differentiation in cancer cells and differentiation therapy of cancers. These results confirm that in contrast to previous toxic methods of treating cancer cells with GBL derivatives, GBL derivatives can be used at subapoptotic concentrations using a method that induces differentiation in cancer cells so as to treat cancers with minimal side effects.

Without limitation, potential subject matter that may be claimed (prefaced with the letter “P” so as to avoid confusion with the actual claims presented below) includes:

P1. A method for treating a human having a cancer, the method comprising: contacting a cancer cell of the human, said cancer cell being associated with the cancer, with a subapoptotic concentration of a gamma butyrolactone derivative, or salt thereof, that induces differentiation in the cancer cell, the gamma butyrolactone derivative having the formula:

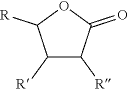

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

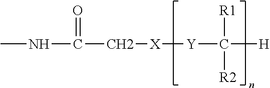

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
         (i) if Y is a single bond, R1 and R2 are each present,
         (ii) if Y is a double bond, R1 is present and R2 is absent, and
         (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

P2. The method of claim P1, wherein a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

P3. The method of claim P1, wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and
wherein none of R, R′, and R″ are N-homoserine.

P4. The method according to any one of claims P1 and P2, wherein the gamma butyrolactone derivative is HSL-C8, HSL-C8 having the formula:

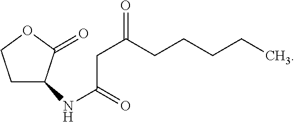 [SMILES: CCCCCC(=O)CC(=O)N[C@H]1CCOC1=O]

P5. The method according to any one of claims P1 and P2, wherein the gamma butyrolactone derivative is HSL-C12, HSL-C12 having the formula:

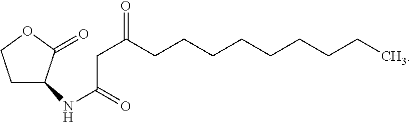 [SMILES: CCCCCCCCCC(=O)CC(=O)N[C@H]1CCOC1=O]

P6. The method according to any one of claims P1 and P3, wherein the gamma butyrolactone derivative is GBL, GBL having the formula:

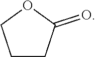

P7. The method according to any one of claims P1 and P3, wherein the gamma butyrolactone derivative is A-factor, A-factor having the formula:

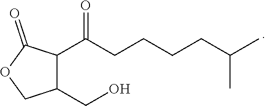 [SMILES: CC(C)CCCCC(=O)C1C(=O)OCC1CO]

P8. The method according to any one of claims P1-P8, wherein the cancer is selected from the group consisting of sarcomas, carcinomas, and leukemias.

P9. The method according to any one of claims P1-P7, wherein the cancer is selected from the group consisting of fibrosarcoma, myxosarcoma, liposarcoma, chondrosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystandenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bluffer carcinoma, epithelial carcinoma, glioma, astrocytomoma, pinealoma, hemangioblastoma, acoustic neuroma, oligodendroglioma, meningioma, melanoma, neuroblastoma, and retinoblastoma.

P10. The method according to any one of claims P1-P7, wherein the cancer is myeloid leukemia.

P11. The method of claim P10, wherein the myeloid leukemia is selected from the group consisting of acute promyelocytic leukemia and acute myeloid leukemia.

P12. The method according to any one of claims P10-P11, wherein the induced differentiation is marked by an increase in the expression of a gene selected from the group consisting of FLT3, PU1, CEBPA, and combinations thereof.

P13. The method according to any one of claims P10-P12, wherein the induced differentiation is marked by an increase in the number of cancer cells expressing a marker selected from the group consisting of CD3, CD20, CD24, CD33, CD16, CD56, and combinations thereof.

P14. A method for inducing differentiation in a cancer cell, said cancer cell being associated with a cancer, the method comprising:
contacting the cancer cell with a subapoptotic concentration of a gamma butyrolactone derivative, or salt thereof, that induces differentiation in the cancer cell, the gamma butyrolactone derivative having the formula:

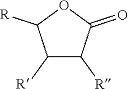

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

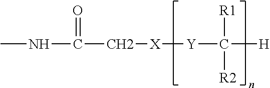

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
         (i) if Y is a single bond, R1 and R2 are each present,
         (ii) if Y is a double bond, R1 is present and R2 is absent, and
         (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, and 11.

P15. The method of claim P14, wherein a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

P16. The method of claim P14, wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and wherein none of R, R′, and R″ are N-homoserine.

P17. The method according to any one of claims P14 and P15, wherein the gamma butyrolactone derivative is HSL-C8, HSL-C8 having the formula:

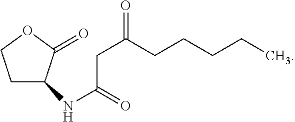 [SMILES: CCCCCC(=O)CC(=O)N[C@H]1CCOC1=O]

P18. The method according to any one of claims P14 and P15, wherein the gamma butyrolactone derivative is HSL-C12, HSL-C12 having the formula:

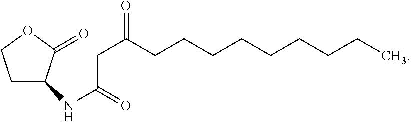 [SMILES: CCCCCCCCCC(=O)CC(=O)N[C@H]1CCOC1=O]

P19. The method according to any one of claims P14 and P16, wherein the gamma butyrolactone derivative is GBL, GBL having the formula:

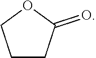

P20. The method according to any one of claims P14 and P16, wherein the gamma butyrolactone derivative is A-factor, A-factor having the formula:

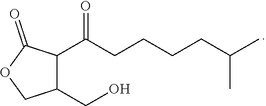 [SMILES: CC(C)CCCCC(=O)C1C(=O)OCC1CO]

P21. The method according to any one claims P14-P20, wherein the cancer is selected from the group consisting of sarcomas, carcinomas, and leukemias.

P22. The method according to any one of claims P14-P20, wherein the cancer is selected from the group consisting of fibrosarcoma, myxosarcoma, liposarcoma, chondrosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystandenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bluffer carcinoma, epithelial carcinoma, glioma, astrocytomoma, pinealoma, hemangioblastoma, acoustic neuroma, oligodendroglioma, meningioma, melanoma, neuroblastoma, and retinoblastoma.

P23. The method according to any one of claims P14-P20, wherein the cancer is myeloid leukemia.

P24. The method of claim P23, wherein the myeloid leukemia is selected from the group consisting of acute promyelocytic leukemia and acute myeloid leukemia.

P25. The method according to any one of claims P23-P24, wherein the induced differentiation is marked by an increase in the expression of a gene selected from the group consisting of FLT3, PU1, CEBPA, and combinations thereof.

P26. The method according to any one of claims P23-P25, wherein the induced differentiation is marked by an increase in the number of cancer cells expressing a marker selected from the group consisting of CD3, CD20, CD24, CD33, CD16, CD56, and combinations thereof.

P27. A method for treating a human having a neoplasm, the method comprising:
contacting a neoplastic cell of the human, said neoplastic cell being associated with the neoplasm, with a subapoptotic concentration of a gamma butyrolactone derivative, or salt thereof, that induces differentiation in the neoplastic cell, the gamma butyrolactone derivative having the formula:

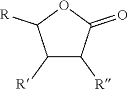

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

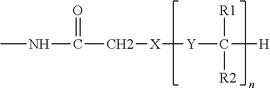

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
      (i) if Y is a single bond, R1 and R2 are each present,
      (ii) if Y is a double bond, R1 is present and R2 is absent, and
      (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

P28. The method of claim P27, wherein a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

P29. The method of claim P27, wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and wherein none of R, R′, and R″ are N-homoserine.

P30. The method according to any one of claims P27 and P28, wherein the gamma butyrolactone derivative is HSL-C8, HSL-C8 having the formula:

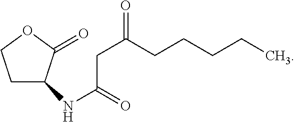 [SMILES: CCCCCC(=O)CC(=O)N[C@H]1CCOC1=O]

P31. The method according to any one of claims P27 and P28, wherein the gamma butyrolactone derivative is HSL-C12, HSL-C12 having the formula:

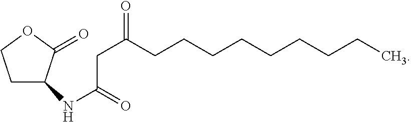 [SMILES: CCCCCCCCCC(=O)CC(=O)N[C@H]1CCOC1=O]

P32. The method according to any one of claims P27 and P29, wherein the gamma butyrolactone derivative is GBL, GBL having the formula:

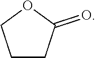

P33. The method according to any one of claims P27 and P29, wherein the gamma butyrolactone derivative is A-factor, A-factor having the formula:

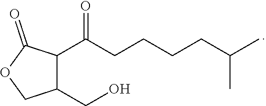 [SMILES: CC(C)CCCCC(=O)C1C(=O)OCC1CO]

P34. The method according to any one of claims P27-P33, wherein the neoplasm is a cancer.

P35. The method of claim P34, wherein the cancer is selected from the group consisting of a a breast cancer, a skin cancer, a thyroid cancer, a urinary system cancer, a reproductive organ cancer, a lung cancer, an epithelial carcinoma, a central or peripheral neural system cancer, and mesothelioma.

P36. The method of claim P34, wherein the cancer is a blood or lymphatic system cancer selected from the group consisting of acute lymphoblastic leukemia, chronic lymphoblastic leukemia, acute myeloid leukemia, acute promyelocytic leukemia, chronic myeloid leukemia, Hodgkin lymphoma, non-Hodgkin lymphoma, diffuse large B-cell lymphoma, lymphangiosarcoma, lymphangioendotheliosarcoma, and endotheliosarcoma.

P37. The method of claim P34, wherein the cancer is a musculoskeletal cancer selected from the group consisting of synovial carcinoma, chondrosarcoma, Ewing's tumor, leiomyosarcoma, myosarcoma, rhabdomyosarcoma, liposarcoma, and fibrosarcoma.

P38. The method of claim P34, wherein the cancer is a gastrointestinal cancer selected from the group consisting of esophageal carcinoma, pancreatic adenocarcinoma, stomach adenocarcinoma, colon adenocarcinoma, and bile duct carcinoma.

P39. The method of claim P34, wherein the cancer is a breast cancer selected from the group consisting of ductal carcinoma, lobular carcinoma, tubular/cribriform carcinoma, mucinous carcinoma, medullary carcinoma, and papillary carcinoma.

P40. The method of claim P34, wherein the cancer is a skin cancer selected from the group consisting of squamous cell carcinoma, basal cell carcinoma, melanoma, sweat gland carcinoma, and sebaceous gland carcinoma.

P41. The method of claim P34, wherein the cancer is a thyroid cancer selected from the group consisting of papillary thyroid carcinoma, medullary thyroid carcinoma, follicular thyroid carcinoma, and anaplastic thyroid carcinoma.

P42. The method of claim P34, wherein the cancer is a urinary system cancer selected from the group consisting of renal cell carcinoma, Wilms' tumor, and urothelial carcinoma.

P43. The method of claim P34, wherein the cancer is a reproductive organ cancer selected from the group consisting of choriocarcinoma, seminoma, embryonal carcinoma, cervical cancer, testicular tumor, prostate cancer, and ovarian cancer.

P44. The method of claim P34, wherein the cancer is a lung cancer selected from the group consisting of lung adenocarcinoma, small cell lung carcinoma, lung squamous cell carcinoma, lung large cell carcinoma, papillary adenocarcinomas, and bronchogenic carcinoma.

P45. The method of claim P34, wherein the cancer is an epithelial carcinoma.

P46. The method of claim P34, wherein the cancer is a central or peripheral neural system cancer selected from the group consisting of glioma, astrocytomoma, pinealoma, hemangioblastoma, acoustic neuroma, oligodendroglioma, meningioma, neuroblastoma, and retinoblastoma.

P47. The method of claim P34, wherein the cancer is mesothelioma.

P48. The method of claim P34, wherein the cancer is a myeloid leukemia.

P49. The method of claim P48, wherein the myeloid leukemia is selected from the group consisting of acute promyelocytic leukemia and acute myeloid leukemia.

P50. The method according to any one of claims P48-P49, wherein the induced differentiation is marked by an increase in the expression of a gene selected from the group consisting of FLT3, PU1, CEBPA, and combinations thereof.

P51. The method according to any one of claims P48-P50, wherein the induced differentiation is marked by an increase in the number of cancer cells expressing a marker selected from the group consisting of CD3, CD20, CD24, CD33, CD16, CD56, and combinations thereof.

P52. The method according to any one of claims P27-P33, wherein the neoplasm is a benign neoplasm.

P53. The method of claim P52, wherein the benign neoplasm is selected from the group consisting of an adenoma, a lipoma, a leiomyoma, a rhabdomyoma, a fibroid, a hemangioma, a meningioma, a neuroma, a schwannoma, a papilloma, a hamartoma, a chondroma, a synovioma, an osteoma, a desmoid tumor, and a hepatoma.

P54. A method for inducing differentiation in a neoplastic cell, said neoplastic cell being associated with a neoplasm, the method comprising:
contacting the neoplastic cell with a subapoptotic concentration of a gamma butyrolactone derivative, or salt thereof, that induces differentiation in the cancer cell, the gamma butyrolactone derivative having the formula:

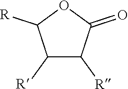

wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula:

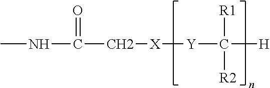

wherein:
   (a) X is selected from the group consisting of C═O, C═S, C═NH, CHOH, CHSH, C, CH, and CH₂,
   (b) Y, at each occurrence in the N-homoserine, is selected from the group consisting of a single bond, a double bond, and a triple bond,
      wherein,
      (i) if Y is a single bond, R1 and R2 are each present,
      (ii) if Y is a double bond, R1 is present and R2 is absent, and
      (iii) if Y is a triple bond, R1 and R2 are each absent; and
   (c) R1, at each occurrence in the N-homoserine, is independently selected from the group consisting of H, an acyl chain, and an alkyl group,
   (d) R2, at each occurrence in the N-homoserine, is independently selected from the group consisting of H, an acyl chain, and an alkyl group, and
   (e) n is an integer selected from the group consisting of 1, 2, 3, 4, 5, 6, 7, 8, 9, 10, and 11.

P55. The method of claim P54, wherein a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

P56. The method of claim P54, wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and wherein none of R, R′, and R″ are N-homoserine.

P57. The method according to any one of claims P54 and P55, wherein the gamma butyrolactone derivative is HSL-C8, HSL-C8 having the formula:

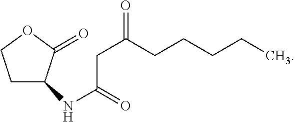 [SMILES: CCCCCC(=O)CC(=O)N[C@H]1CCOC1=O]

P58. The method according to any one of claims P54 and P55, wherein the gamma butyrolactone derivative is HSL-C12, HSL-C12 having the formula:

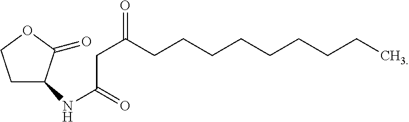 [SMILES: CCCCCCCCCC(=O)CC(=O)N[C@H]1CCOC1=O]

P59. The method according to any one of claims P54 and P56, wherein the gamma butyrolactone derivative is GBL, GBL having the formula:

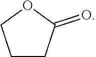

P60. The method according to any one of claims P54 and P56, wherein the gamma butyrolactone derivative is A-factor, A-factor having the formula:

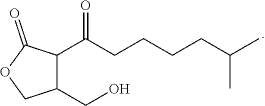 [SMILES: CC(C)CCCCC(=O)C1C(=O)OCC1CO]

P61. The method according to any one of claims P54-P60, wherein the neoplasm is a cancer.

P62. The method of claim P61, wherein the cancer is selected from the group consisting of a a breast cancer, a skin cancer, a thyroid cancer, a urinary system cancer, a reproductive organ cancer, a lung cancer, an epithelial carcinoma, a central or peripheral neural system cancer, and mesothelioma.

P63. The method of claim P61, wherein the cancer is a blood or lymphatic system cancer selected from the group consisting of acute lymphoblastic leukemia, chronic lymphoblastic leukemia, acute myeloid leukemia, acute promyelocytic leukemia, chronic myeloid leukemia, Hodgkin lymphoma, non-Hodgkin lymphoma, diffuse large B-cell lymphoma, lymphangiosarcoma, lymphangioendotheliosarcoma, and endotheliosarcoma.

P64. The method of claim P61, wherein the cancer is a musculoskeletal cancer selected from the group consisting of synovial carcinoma, chondrosarcoma, Ewing's tumor, leiomyosarcoma, myosarcoma, rhabdomyosarcoma, liposarcoma, and fibrosarcoma.

P65. The method of claim P61, wherein the cancer is a gastrointestinal cancer selected from the group consisting of esophageal carcinoma, pancreatic adenocarcinoma, stomach adenocarcinoma, colon adenocarcinoma, and bile duct carcinoma.

P66. The method of claim P61, wherein the cancer is a breast cancer selected from the group consisting of ductal carcinoma, lobular carcinoma, tubular/cribriform carcinoma, mucinous carcinoma, medullary carcinoma, and papillary carcinoma.

P67. The method of claim P61, wherein the cancer is a skin cancer selected from the group consisting of squamous cell carcinoma, basal cell carcinoma, melanoma, sweat gland carcinoma, and sebaceous gland carcinoma.

P68. The method of claim P61, wherein the cancer is a thyroid cancer selected from the group consisting of papillary thyroid carcinoma, medullary thyroid carcinoma, follicular thyroid carcinoma, and anaplastic thyroid carcinoma.

P69. The method of claim P61, wherein the cancer is a urinary system cancer selected from the group consisting of renal cell carcinoma, Wilms' tumor, and urothelial carcinoma.

P70. The method of claim P61, wherein the cancer is a reproductive organ cancer selected from the group consisting of choriocarcinoma, seminoma, embryonal carcinoma, cervical cancer, testicular tumor, prostate cancer, and ovarian cancer.

P71. The method of claim P61, wherein the cancer is a lung cancer selected from the group consisting of lung adenocarcinoma, small cell lung carcinoma, lung squamous cell carcinoma, lung large cell carcinoma, papillary adenocarcinomas, and bronchogenic carcinoma.

P72. The method of claim P61, wherein the cancer is an epithelial carcinoma.

P73. The method of claim P61, wherein the cancer is a central or peripheral neural system cancer selected from the group consisting of glioma, astrocytomoma, pinealoma, hemangioblastoma, acoustic neuroma, oligodendroglioma, meningioma, neuroblastoma, and retinoblastoma.

P74. The method of claim P61, wherein the cancer is mesothelioma.

P75. The method of claim P61, wherein the cancer is a myeloid leukemia.

P76. The method of claim P75, wherein the myeloid leukemia is selected from the group consisting of acute promyelocytic leukemia and acute myeloid leukemia.

P77. The method according to any one of claims P75-P76, wherein the induced differentiation is marked by an increase in the expression of a gene selected from the group consisting of FLT3, PU1, CEBPA, and combinations thereof.

P78. The method according to any one of claims P75-P77, wherein the induced differentiation is marked by an increase in the number of cancer cells expressing a marker selected from the group consisting of CD3, CD20, CD24, CD33, CD16, CD56, and combinations thereof.

P79. The method according to any one of claims P54-P60, wherein the neoplasm is a benign neoplasm.

P80. The method of claim P79, wherein the benign neoplasm is selected from the group consisting of an adenoma, a lipoma, a leiomyoma, a rhabdomyoma, a fibroid, a hemangioma, a meningioma, a neuroma, a schwannoma, a papilloma, a hamartoma, a chondroma, a synovioma, an osteoma, a desmoid tumor, and a hepatoma.

The embodiments of the invention described above are intended to be merely exemplary; numerous variations and modifications will be apparent to those skilled in the art. All such variations and modifications are intended to be within the scope of the present invention as defined in any appended claims.

What is claimed is:

1. A method for treating a human having a neoplasm, the method comprising:
contacting a neoplastic cell of the human, said neoplastic cell being associated with the neoplasm, with a subapoptotic concentration of a gamma butyrolactone derivative, or salt thereof, that induces differentiation in the neoplastic cell, the gamma butyrolactone derivative having the formula: 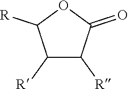
wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula: 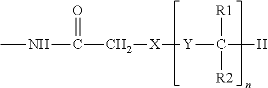
wherein:
(a) X is selected from the group consisting of C═O, C═S, C═NH, CHOH, CHSH, C, CH, and CH₂,
(b) Y, at each occurrence in the N-homoserine, is selected from the group consisting of a single bond, a double bond, and a triple bond,
wherein,
(i) if Y is a single bond, R1 and R2 are each present,
(ii) if Y is a double bond, R1 is present and R2 is absent, and
(iii) if Y is a triple bond, R1 and R2 are each absent; and
(c) R1, at each occurrence in the N-homoserine, is independently selected from the group consisting of H, an acyl chain, and an alkyl group,
(d) R2, at each occurrence in the N-homoserine, is independently selected from the group consisting of H, an acyl chain, and an alkyl group, and
(e) n is an integer selected from the group consisting of 1, 2, 3, 4, 5, 6, 7, 8, 9, 10, and 11.

2. The method of claim 1, wherein a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

3. The method of claim 1, wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and
wherein none of R, R′, and R″ are N-homoserine.

4. The method of claim 1, wherein the gamma butyrolactone derivative is HSL-C8, HSL-C8 having the formula:

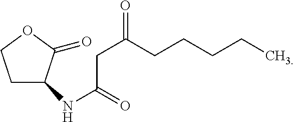 [SMILES: CCCCCC(=O)CC(=O)N[C@H]1CCOC1=O]

5. The method according of claim 1, wherein the gamma butyrolactone derivative is HSL-C12, HSL-C12 having the formula:

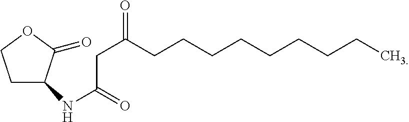 [SMILES: CCCCCCCCCC(=O)CC(=O)N[C@H]1CCOC1=O]

6. The method of claim 1, wherein the gamma butyrolactone derivative is GBL, GBL having the formula:

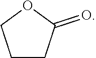

7. The method of claim 1, wherein the gamma butyrolactone derivative is A-factor, A-factor having the formula:

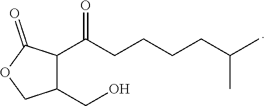 [SMILES: CC(C)CCCCC(=O)C1C(=O)OCC1CO]

8. The method of claim 1, wherein the neoplasm is:
(i) a cancer selected from the group consisting of a blood cancer, a lymphatic system cancer, a musculoskeletal cancer, a gastrointestinal cancer, a breast cancer, a skin cancer, a thyroid cancer, a urinary system cancer, a reproductive organ cancer, a lung cancer, an epithelial carcinoma, a central or peripheral neural system cancer, and mesothelioma; or
(ii) a benign neoplasm selected from the group consisting of an adenoma, a lipoma, a leiomyoma, a rhabdomyoma, a fibroid, a hemangioma, a meningioma, a neuroma, a schwannoma, a papilloma, a hamartoma, a chondroma, a synovioma, an osteoma, a desmoid tumor, and a hepatoma.

9. The method of claim 1, wherein the neoplasm is a myeloid leukemia selected from the group consisting of acute promyelocytic leukemia and acute myeloid leukemia, and
wherein the induced differentiation is marked by an increase in the expression of a gene selected from the group consisting of FLT3, PU1, CEBPA, and combinations thereof.

10. The method of claim 1, wherein the neoplasm is a myeloid leukemia selected from the group consisting of acute promyelocytic leukemia and acute myeloid leukemia, and
wherein the induced differentiation is marked by an increase in the number of cancer cells expressing a marker selected from the group consisting of CD3, CD20, CD24, CD33, CD16, CD56, and combinations thereof.

11. A method for inducing differentiation in a neoplastic cell, said neoplastic cell being associated with a neoplasm, the method comprising:
contacting the neoplastic cell with a subapoptotic concentration of a gamma butyrolactone derivative, or salt thereof, that induces differentiation in the cancer cell, the gamma butyrolactone derivative having the formula: 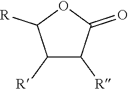
wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, ester, and N-homoserine,
N-homoserine having the formula: 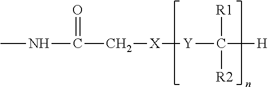
wherein:
(a) X is selected from the group consisting of C═O, C═S, C═NH, CHOH, CHSH, C, CH, and CH₂,
(b) Y, at each occurrence in the N-homoserine, is selected from the group consisting of a single bond, a double bond, and a triple bond,
wherein,
(i) if Y is a single bond, R1 and R2 are each present,
(ii) if Y is a double bond, R1 is present and R2 is absent, and
(iii) if Y is a triple bond, R1 and R2 are each absent; and
(c) R1, at each occurrence in the N-homoserine, is independently selected from the group consisting of H, an acyl chain, and an alkyl group,
(d) R2, at each occurrence in the N-homoserine, is independently selected from the group consisting of H, an acyl chain, and an alkyl group, and
(e) n is an integer selected from the group consisting of 1, 2, 3, 4, 5, 6, 7, 8, 9, 10, and 11.

12. The method of claim 11, wherein a corresponding one of R, R′, and R″ is N-homoserine, and wherein R, R′, and R″, other than the corresponding one, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester.

13. The method of claim 11, wherein R, R′, and R″, are each independently selected from the group consisting of hydrogen, hydroxyl, NH₂, Se, S, halogen, phenyl, benzyl, carboxylic acid, carbonyl, unsubstituted alkyl, substituted alkyl, unsubstituted alkene, substituted alkene, unsubstituted alkyne, substituted alkyne, unsubstituted aryl, substituted aryl, unsubstituted alkoxy, substituted alkoxy, and ester, and
wherein none of R, R′, and R″ are N-homoserine.

14. The method of claim 11, wherein the gamma butyrolactone derivative is HSL-C8, HSL-C8 having the formula:

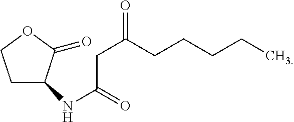 [SMILES: CCCCCC(=O)CC(=O)N[C@H]1CCOC1=O]

15. The method of claim 11, wherein the gamma butyrolactone derivative is HSL-C12, HSL-C12 having the formula:

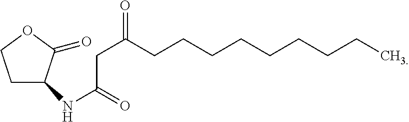 [SMILES: CCCCCCCCCC(=O)CC(=O)N[C@H]1CCOC1=O]

16. The method of claim 11, wherein the gamma butyrolactone derivative is GBL, GBL having the formula:

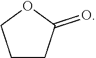

17. The method of claim 11, wherein the gamma butyrolactone derivative is A-factor, A-factor having the formula:

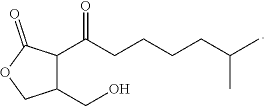 [SMILES: CC(C)CCCCC(=O)C1C(=O)OCC1CO]

18. The method of claim 11, wherein the neoplasm is:
(i) a cancer selected from the group consisting of a blood cancer, a lymphatic system cancer, a musculoskeletal cancer, a gastrointestinal cancer, a breast cancer, a skin cancer, a thyroid cancer, a urinary system cancer, a reproductive organ cancer, a lung cancer, an epithelial carcinoma, a central or peripheral neural system cancer, and mesothelioma; or
(ii) a benign neoplasm selected from the group consisting of an adenoma, a lipoma, a leiomyoma, a rhabdomyoma, a fibroid, a hemangioma, a meningioma, a neuroma, a schwannoma, a papilloma, a hamartoma, a chondroma, a synovioma, an osteoma, a desmoid tumor, and a hepatoma.

19. The method of claim 11, wherein the neoplasm is a myeloid leukemia selected from the group consisting of acute promyelocytic leukemia and acute myeloid leukemia, and
wherein the induced differentiation is marked by an increase in the expression of a gene selected from the group consisting of FLT3, PU1, CEBPA, and combinations thereof.

20. The method of claim 11, wherein the neoplasm is a myeloid leukemia selected from the group consisting of acute promyelocytic leukemia and acute myeloid leukemia, and
wherein the induced differentiation is marked by an increase in the number of cancer cells expressing a marker selected from the group consisting of CD3, CD20, CD24, CD33, CD16, CD56, and combinations thereof.
